SEQUENCE LISTING (6 sequences)

SEQ ID NO: 1 - DNA, 10 bp, Artificial Sequence
  synthetic construct
ttcgctcgta

SEQ ID NO: 2 - DNA, 10 bp, Artificial Sequence
  synthetic construct
ttcgatcgta

SEQ ID NO: 3 - DNA, 10 bp, Artificial Sequence
  synthetic construct
ttcgctcgta

SEQ ID NO: 4 - DNA, 10 bp, Artificial Sequence
  synthetic construct
ttcgatcgta

SEQ ID NO: 5 - DNA, 10 bp, Artificial Sequence
  synthetic construct
ttcgatcgta

SEQ ID NO: 6 - DNA, 28 bp, Artificial Sequence
  synthetic construct
gagtatggaaagaattagttttccgaaa

(19) United States
(12) Patent Application Publication
(10) Pub. No.: US 2023/0141128 A1
(43) Pub. Date: May 11, 2023

(54) MOLECULAR TECHNOLOGY FOR PREDICTING A PHENOTYPIC TRAIT OF A BACTERIUM FROM ITS GENOME

(71) Applicant: BIOMÉRIEUX, Marcy L'Etoile (FR)

(72) Inventors: Magali JAILLARD DANCETTE, Marcy l'Etoile (FR); Pierre MAHE, Lans en Vercors (FR)

(73) Assignee: BIOMÉRIEUX, Marcy L'Etoile (FR)

(21) Appl. No.: 17/910,982

(22) PCT Filed: Mar. 10, 2021
(86) PCT No.: PCT/EP2021/056013

§ 371 Date: Sep. 12, 2022

(30) Foreign Application Priority Data
Mar. 12, 2020 (EP) 20162655.3

(51) Int. Cl. G16B 20/00; G16B 40/20

(52) U.S. Cl. CPC G16B 20/00; G16B 40/20; G16B 30/00

(57) ABSTRACT

A process of determining a phenotypic trait of a bacterial strain comprises sequencing part or all of the genome of the strain, and applying to the sequenced genome a predetermined model for predicting the trait, the model having groups of genome sequences as variables. According to the invention, the groups are chosen so that a co-occurrence rate in the genome of the bacterial species of the constituent genome sequences of the groups is above a predetermined threshold, and the groups are clusterings of the genome sequences according to their co-occurrence rates in the genome of the bacterial species.

### FIELD OF THE INVENTION

The present invention relates to the technical field of molecular biology applied to bacterial genomics, and in particular to the field of predicting phenotypic traits of bacteria from their genomes. The invention is particularly applicable to the prediction of antibiotic susceptibility and virulence of bacteria present in a biological sample.

### PRIOR ART

A. Molecular Technology for Phenotype Prediction

The susceptibility of a bacterial strain to an antibiotic, i.e. its sensitivity or resistance in the context of a treatment based on the antibiotic administered to a human or an animal, cannot be directly observed by a human being. Specifically, direct observation of the strain, even using microscopes, does not make it possible to determine its behavior toward the antibiotic. in vitro diagnosis in a bacterial context consists, by nature, in making this phenotypic nature observable and therefore ultimately exploitable for a clinician. During the 20th century, in vitro diagnostic technologies essentially combined culture-based sample preparation techniques, notably to make the bacterial strains present in the samples visible and manipulable, and techniques for optical measurement of the behavior of the strains in the presence of an antibiotic. For example, a conventional microbiology laboratory workflow involves first spreading a sample from a patient suspected of having a bacterial infection on a culture medium to produce bacterial colonies that are visible to a human operator or an automated system after incubation. In a second stage, when the colonies are large enough, a technician or an automated system takes a colony, mixes it with an antibiotic at different concentrations and introduces the mixtures into a device that measures the optical density of each mixture and deduces therefrom the susceptibility to the antibiotic. Since the optical density indicates the bacterial proliferation, it unambiguously characterizes the sensitivity or resistance of the bacterium: if the density increases, this means that it is proliferating despite the presence of the antibiotic, and thus that it is resistant to the antibiotic at the concentration of the antibiotic under consideration.

Today, the combination of sample preparation technologies and optical density-based measurement technologies has significant limitations in the face of rapid global development in the prokaryotic kingdom, namely the acquisition of multi-drug resistance to antibiotics, which is estimated to be responsible for more deaths than cancer by 2050. First of all, these technologies are not agnostic as regards bacteria. Specifically, depending on the chosen culture medium, certain strains will grow and others will not, and as such these technologies do not make it possible to characterize the antibiotic susceptibility of all bacterial species.

Secondly, these techniques are extremely slow since they are based on bacterial culturing that takes a long time. Thus, obtaining an antibiogram of a bacterium takes at least 30 hours from the time the sample is taken. This delay does not allow effective treatment of patients who are systematically given a broad-spectrum antibiotic cocktail as a first-line treatment. In addition to the consequences for the patient, this inappropriate and massive administration of antibiotics reinforces the selection pressure of multi-drug-resistant bacteria and thus contributes toward their expansion. It is thus believed at the present time that conventional in vitro diagnostic technologies are increasingly unsuitable for treating patients and are, to a certain extent, one of the reasons for the emergence of multi-drug resistance.

The maturation of molecular biology technologies, in particular bacterial DNA and/or RNA characterization technologies, such as polymerase chain reaction (PCR), DNA chips or sequencing, is bringing about a paradigm shift in the analysis of antibiotic resistance in laboratories. Firstly, they are more agnostic toward bacterial species. For example, metagenomic technology makes it possible to process bacterial DNA in a biological sample irrespective of the bacterial species present. Secondly, they aim to provide a result in a few hours, with some, such as PCR, even providing a result in less than 20 minutes. On the other hand, molecular techniques for characterizing antibiotic susceptibility are based on genomic signatures (absence/presence of genes, genetic mutations, predictive models, etc.) characterizing said susceptibility. FIG. 1 illustrates in a simplified and nonlimiting manner two bacterial DNA characterization technologies, namely a PCR technology 10 and a whole genome sequencing (WGS) technology 20 applied in microbiological workflows for the treatment of a patient suspected of having a bacterial infection. Both workflows begin with the collection 12 of a biological sample from the patient, followed by the application of PCR 10 or WGS 20 technology, each yielding a result 106, 210 of genomic signatures characterizing susceptibility to one or more antibiotics, on the basis of which result an antibiotic treatment is chosen and administered to the patient by a clinician in 14. Basically, each of the molecular technologies requires the preparation 102, 202 of the sample taken prior to the application of the PCR itself 104, e.g. a nested PCR performed using a Filmarray platform from the company Biofire, or the application of sequencing 204, e.g. SBS sequencing performed using a MiSeq platform from the company Illumina.

One of the main differences between these two molecular technologies is the nature of the genomic signatures. In the case of PCR, the genomic signature is molecular, and therefore tangible: it is translated into primers introduced into a reaction mixture, these primers specifically targeting bacterial DNA sequences introduced into the mixture, and its detection is generally achieved by measuring an optical signal. In contrast, in the case of WGS, genomic signatures are digital since the sequencing produces a digital genome and the processing of said genome is computerized. Whereas the WGS technology allows, at the very least, the implementation of PCR genomic signatures, it above all allows complex exploitation of the digital genome and the use of predictive models of antibiotic susceptibility that are impossible to implement using PCR technologies. Thus, the WGS technology is advantageously based on a computer design of the genomic signatures 30, this design advantageously exploiting large genomic and phenotypic knowledge bases with the aid of complex analytical tools, for example machine learning technologies such as parsimoniously constrained logistic regressions.

This being the case, all molecular techniques are based on the same technical foundation, namely the measurement of genomic information from bacterial strains and the processing of said information so as to extract information regarding the behavior of the strains in the presence of an antibiotic. Moreover, while these computer technologies, and more particularly those of sequencing, are different in nature from those associated with optical density analysis of more conventional microbiological technologies, they do not change the technical nature of the in vitro diagnosis they perform. For example, in the case of infectious diagnostics, it is still a matter of applying technologies for processing a biological sample so as to determine whether a patient has a bacterial infection and to know the behavior of the infectious bacterium in the presence of an antibiotic in order to administer an appropriate antibiotic treatment.

B. Interpretability of Molecular Technologies for Phenotype Prediction

Focusing more particularly on genomic signatures, the first approaches consisted in identifying previously identified antibiotic resistance markers in the bacterial genome, referred to as “direct association” approaches. While these approaches are effective when the genetic mechanisms leading to resistance are well known and simple, as is the case for most resistance mechanisms in species such as Mycobacterium tuberculosis, they suffer from major limitations: incomplete knowledge of resistance mechanisms in many species and antibiotics, resulting, for example, in incomplete databases, difficulty in taking into account differences in the predictive powers of markers and the multifactorial aspect of antibiotic susceptibility (e.g. epistasis, combination of multiple mutations, etc.), etc. Faced with these difficulties, the genetic determinism of antibiotic susceptibility is being addressed more effectively by new approaches based on advanced computer technologies, and in particular by supervised machine learning technologies, the learning and application architecture of which can be summarized as follows:
A. for a set of learning bacterial strains:
   a. each strain is sequenced and phenotypically characterized (e.g. measurement of its minimum inhibitory concentration and/or measurement of its susceptibility—resistance, intermediate or sensitive—to one or more antibiotics);
   b. a computer model for predicting the susceptibility to the antibiotic is trained from genomes and phenotypic data.
B. for a new strain whose susceptibility to an antibiotic from step (A. a) is sought:
   a. the strain is sequenced;
   b. the computer prediction model is applied to its digital genome so as to determine its susceptibility.

The above generic description involves first defining the machine learning variables that describe the bacterial genome. There are many ways to describe said genome, one of them being the description in “k-mers”, i.e. the list of nucleic acid sequences of length k (i.e. number of bases) constituting the genome. As described in M. Jaillard Dancette's thesis, “Vers une cartographie des polymorphisms liés à la résistance aux antimicrobiens [Toward a mapping of polymorphisms related to antimicrobial resistance]”, 2018, this description is particularly suited to bacterial genomes which are haploid and highly plastic compared to eukaryotic genomes. In other words, this description effectively describes the diversity of genetic mechanisms underlying antibiotic susceptibility in bacteria.

However, this description has a number of drawbacks that may have a negative impact on machine learning technologies, including the following:
a. k-mers are highly redundant: those covering a conserved genomic region can be co-occurring, i.e. present or absent in a systematic manner in a set of genomes, and are therefore statistically equivalent;
b. some k-mers are not specific to a genomic region and as such they are difficult to annotate, i.e. to characterize structurally or functionally (gene, mutation, etc.);
c. the genome-susceptibility association is a very high-dimensional problem, the number of k-mers per genome being greater than a hundred thousand or even a million, and as such redundancy and non-specificity lead to highly correlated variables for the learning tools.

For high-risk fields, particularly that of human health, it is important to reduce the dimensionality of the variables to increase the interpretability of prediction models. Specifically, machine learning tools are sensitive to biases in the learning data, for example lack of genomic diversity, and to biases associated with an incomplete formulation of susceptibility as a function of the bacterial genome, for example failure to take into account strong correlations between different genomic regions. By reducing the dimensionality, prediction models become easier to interpret for both learning tool experts and bacterial genomics experts, enabling biases to be detected and thus enabling the construction of appropriate learning data or reformulating the problem that the learning tool is intended to solve. Similarly, since they are more interpretable, prediction models are easier to validate for use in a high-risk field if no biases are apparent after their analysis.

Among the tools that allow a strong reduction in dimensionality, parsimonious automatic learning tools, for instance penalized lasso regressions or decision tree-based tools, make it possible to obtain a number of predictive k-mers, i.e. k-mers that are retained in the prediction model, of the order of a thousand or even a hundred. However, these tools are unstable in a high-dimensional environment in which the variables are highly correlated. Thus, they can select predictor variables that together form a genomic unit that does not necessarily have any biological reality, and as such the prediction models remain difficult to interpret. Certain techniques can take into account strong correlations between variables, notably elastic-net penalty-based regressions which combine L1-type penalties with L2-type penalties, leading to the selection of groups of correlated predictor variables. It should be noted, however, that this clustering remains mainly algorithmic and that the groups of variables retained are still difficult to interpret biologically.

Other tools, for instance group-lasso tools, enable the a priori clustering of descriptive variables into genomic units. In this context, all the variables in a unit are either predictive or not, depending on the selection or non-selection of the unit by the group-lasso strategy. However, since the description in k-mers is difficult to interpret for the reasons mentioned above, the a priori definition in explanatory units is difficult. In particular, this assumes that high correlation phenomena in a high-dimensional space are well understood and formalized by experts in bacterial genomics, since a lack of knowledge, or imperfect knowledge of these phenomena, translates into bias in the machine learning algorithm.

C. Application of Molecular Prediction Technologies to Biological Variability

In parallel to the problem presented above, when a prediction is based on groups of genome sequences, the question arises as to whether a group is present in the genome of a strain if all the sequences that make up the cluster are all present in the genome in an identical manner. If such a criterion is applied, it assumes that the learning data are complete to encompass all the genomic variability of the bacterial species. In addition to the fact that it is difficult to judge the completeness of the learning data at a given time, said data very often become incomplete over time in certain bacterial species due to the very significant plasticity of their genome. Also, the application of a criterion that is too strict leads to a high rate of false positives or false negatives.

The problems which have just been described in relation to the genomic prediction of the susceptibility of a bacterial strain to one or more antibiotics arise in the same terms for any genomic determination of a phenotypic trait of the strain, for example its virulence or its ribotype.

### DESCRIPTION OF THE INVENTION

The aim of the present invention is to provide a genomic prediction of a phenotypic trait, in particular of the susceptibility to an antibiotic, which is more representative of the biological reality of said trait of the bacterial species to which the strain belongs while at the same time maintaining the performance of the prediction.

To this end, one subject of the invention is a process for determining a phenotypic trait of a bacterial strain, involving:
partial or total sequencing of the genome of said strain;
applying to the sequenced genome a predetermined model for predicting said trait, said model having groups of genome sequences as variables.

According to the invention, said groups are chosen so that:
a co-occurrence rate, in the genomes of the bacterial species to which said strain belongs, of the constituent genome sequences of said groups is greater than a predetermined threshold; and
the groups are clusterings of said genome sequences according to their co-occurrence rates in the genomes of the bacterial species.

In other words, the invention forces machine learning to consider genome sequences of equivalent weight in the prediction of the phenotypic trait as groups rather than as independent variables. The inventors thus noted that the prediction does not suffer a degradation in performance while, at the same time, the learned group(s) that have the most predictive weight correspond to a biological reality, for example correspond to whole genes or groups of specific mutations. This approach has been validated notably on bacterial species whose genomic origin of susceptibility is well documented.

In the text hereinbelow, an application to antibiotic susceptibility is described, it being understood that the application also applies to any phenotypic trait.

According to one embodiment of the invention, a first subset of the genome sequences is a set of unclustered variables of a first susceptibility prediction model. In other words, this filtering makes it possible to eliminate from the outset the sequences that have no predictive weight by advantageously using unconstrained learning tools for clustering, which are thus a priori the most efficient.

In particular, the first prediction model is a model trained on a database of genomes and susceptibilities of bacterial strains belonging to the bacterial species, the learning belonging to the class of parsimonious learning, advantageously a logistic regression, notably of the LASSO type. The term “parsimonious learning” means learning that defines a prediction model whose variables form a subset of the descriptive variables of a problem, directed toward minimizing the size of said subset without having an impact on the prediction performance of the model.

In other words, parsimonious approaches allow the selection of a first set of highly predictive genome sequences which will be joined by other sequences that have very similar co-occurrence units. More particularly, a parsimonious approach can identify regions of the genome that are involved in antibiotic susceptibility. Advantageously, a second subset of the genome sequences is a set of genome sequences having a co-occurrence rate with the first set above the predetermined threshold. In other words, while parsimonious approaches allow an initial identification of genomic regions of interest, they generally fail, due to correlations between variables, to exhaustively describe said regions. By extending the first set of sequences obtained by parsimonious learning with a second set of co-occurring sequences, a set of sequences allowing an exhaustive description of said regions is generated.

According to one embodiment, the genome sequences correspond to nodes of a compacted graph obtained from genome sequences of constant length, of length k in number of bases, or “k-mers”, and occurring in the genomes of strains belonging to the bacterial species. In particular, said compacted graph is calculated by a) calculating a De Bruijn graph of alphabet A, T, G, C from said genome sequences of constant length, b) compacting linear paths of the De Bruijn graph in order to obtain said compacted graph. In this manner, a set of unique genome sequences is obtained, the redundancy existing in the descriptive sequences of constant length, namely the k-mers, being eliminated.

According to one embodiment, the clustering of the genome sequences involves:
obtaining first groups of genome sequences by applying sequence clustering with a perfect co-occurrence in genomes of strains belonging to the bacterial species;
obtaining the groups of the prediction model by applying a second clustering of said first groups based on the co-occurrence rates of said first groups in said genomes.

In this manner, sequences that are systematically present at the same time in each of the genomes of the strains of the bacterial species which was used for constructing the compacted graph, or absent at the same time, or some present and some absent at the same time, are clustered into single genomic units, reducing the number of variables, and also a hidden redundancy.

According to one embodiment, the value of a group of genome sequences for the application of the prediction model applied to the bacterial strain is equal to the percentage of said first groups of said group that are present in the genome of the bacterial strain (328).

According to one embodiment, the value of a group of genome sequences for the application of the prediction model applied to the bacterial strain is calculated by:
calculating, for each first group of said group, the percentage of genome sequences of said first group present in the genome of the bacterial strain; and
calculating the value as being equal to the mean of the percentages of the first groups of said group.

According to one embodiment, the clustering of genome sequences involves a) calculating a dendrogram based on the co-occurrence rates of genome sequences in genomes of strains belonging to the bacterial species, and b) clustering the dendrogram at a predetermined height so as to obtain the groups of genome sequences. In particular, the dendrogram is calculated on the first groups of genome sequences. This occurrence-based hierarchical approach allows the number of sequence groups to be comprehensibly adjusted. By adjusting the cluster height, it is possible to optimize the biological value of the selected groups.

According to one embodiment, the prediction model is trained on a database of genomes and susceptibilities of bacterial strains belonging to the bacterial species, the learning algorithm belonging to the parsimonious learning class, in particular based on logistic regression. In particular, the learning is performed by logistic regression, notably of the LASSO type. By the parsimonious approach, the most predictive groups are selected, these groups constituting genomic signatures of susceptibility.

Advantageously, the value of a group of genome sequences for parsimonious learning is calculated by
for each genome sequence belonging to said group, calculating a vector of occurrences of said sequence in the genomes of the database;
calculating the mean vector of the occurrence vectors, said mean vector constituting the value of said group.

According to one embodiment, the value of a group of genome sequences for the prediction model applied to the bacterial strain is equal to the percentage of genome sequences of said group present in the genome of the bacterial strain. In particular, a genome sequence is present in the genome of the bacterial strain when a percentage of genome sequences of constant length constituting said sequence is higher than a predetermined threshold.

A subject of the invention is also a process for identifying genomic signatures that are predictive of antibiotic susceptibility of a bacterial species, the process involving:
building a database of genomes and susceptibility of a plurality of bacterial strains belonging to the bacterial species;
calculating a set of genome sequences that are descriptive of the genomes in the database, and for each of said sequences calculating a vector of occurrence of said sequence in the genomes in the database;
selecting, from said set of genome sequences, sequences having a co-occurrence rate higher than a predetermined threshold, said rate being calculated according to the occurrence vectors;
clustering the chosen genome sequences according to their co-occurrence rates, so as to obtain groups of genome sequences;
training a model for predicting the susceptibility of the bacterial species to the antibiotic with the groups of genome sequences as variables;
identifying the signature as being equal to the union of groups of genome sequences.

A subject of the invention is also a use of a genomic signature identified according to a process for identifying genomic signatures of the abovementioned type, in a molecular test for predicting a phenotypic trait, in particular susceptibility to an antibiotic, in particular a polymerase chain reaction type test, or a DNA chip-based test, said test at least partially targeting the genomic signature.

A subject of the invention is also a computer program product storing computer-executable instructions for applying a model for predicting a phenotypic trait, in particular the susceptibility to an antibiotic, of a bacterial strain of the abovementioned type.

A subject of the invention is also a system for determining the susceptibility to an antibiotic of a bacterial strain, comprising:
a sequencing platform for partial or total sequencing of the genome of said strain;
a computer unit configured for applying, to the sequenced genome, a predetermined model for predicting a phenotypic trait of the abovementioned type.

A subject of the invention is also a computer program product storing computer-executable instructions for performing a process for identifying genomic signatures predictive of a phenotypic trait, in particular of the susceptibility to an antibiotic, of a bacterial strain of the abovementioned type.

### BRIEF DESCRIPTION OF THE FIGURES

The invention will be better understood on reading the following description, which is given purely by way of example, and made in relation to the appended drawings, in which identical references denote identical or similar elements, and in which:

FIG. 1 illustrates two molecular technologies of the prior art for predicting the susceptibility of a bacterial strain to an antibiotic;

FIGS. 2A and 2B are flow charts of a learning phase and a prediction phase according to the invention;

FIG. 3 illustrates MAF unit generation according to the invention;

FIG. 4 illustrates a clustering used in the process according to the invention;

FIG. 5 is an ROC curve illustrating the selection of a prediction threshold according to the invention;

FIG. 6 illustrates, for the bacterial species Klebsiella pneumoniae and the antibiotic meropenem, the coefficients of a prediction model obtained according to a lasso technique and according to the process of the invention, named “cluster-lasso”, and also the location of the predictive variables of two lasso and cluster-lasso models in the sub-graph of the compacted graph annotated as being blaKPC gene;

FIG. 7 illustrates, for the bacterial species K. pneumoniae and the antibiotic cefoxitin, the subgraph(s) of the compacted graph involving the most predictive extended MAF units of the lasso model (left part), and the subgraph(s) of the compacted graph involving the most predictive clusters of the cluster-lasso model (right-hand part);

FIG. 8 illustrates, for the species Salmonella and for the antibiotic tetracycline, the absolute values of the coefficients of the lasso model, the absolute values of the coefficients of the cluster-lasso model, and the number of unitigs included in the first 10 most predictive clusters of the cluster-lasso model;

FIG. 9 illustrates, for the species Salmonella and for the antibiotic tetracycline, the subgraph(s) of the compacted graph involving the most predictive extended MAF units of the lasso model (left part), and the subgraph(s) of the compacted graph involving the most predictive clusters of the cluster-lasso model (right-hand part);

FIG. 10 shows, for the species Salmonella and for the antibiotic gentamicin, the absolute values of the coefficients of the lasso model, the absolute values of the coefficients of the cluster-lasso model, and the number of unitigs included in the first 10 most predictive clusters of the cluster-lasso model;

FIG. 11 illustrates, for the species Salmonella and for the antibiotic gentamicin, the subgraph(s) of the compacted graph involving the most predictive extended MAF units of the lasso model (left part), and the subgraph(s) of the compacted graph involving the most predictive clusters of the cluster-lasso model (right-hand part);

FIG. 12 illustrates, for the species Neisseria gonorrhoeae and for the antibiotic cefixime, the sub-graph(s) of the compacted graph involving the most predictive extended MAF units of the lasso model (left part), and the sub-graph(s) of the compacted graph involving the most predictive clusters of the cluster-lasso model (right-hand part);

FIG. 13 illustrates, for the species Neisseria gonorrhoeae and for the antibiotic cefixime, the absolute values of the coefficients of the lasso model, the absolute values of the coefficients of the cluster-lasso model, and the number of unitigs included in the first 10 most predictive clusters of the cluster-lasso model;

FIG. 14 illustrates for the species Staphylococcus aureus and for the antibiotic tetracycline, the sub-graph(s) of the compacted graph involving the most predictive extended MAF units of the lasso model (left part), and the sub-graph(s) of the compacted graph involving the most predictive clusters of the cluster-lasso model (right-hand part);

FIG. 15 shows, for the species Staphylococcus aureus and for the antibiotic tetracycline, the absolute values of the coefficients of the lasso model, the absolute values of the coefficients of the cluster-lasso model, and the number of unitigs included in the first 10 most predictive clusters of the cluster-lasso model;

FIG. 16 illustrates a flow chart for detecting a genome sequence in a genome;

FIG. 17 shows the breakdown of the genome sequence into k-mers;

FIG. 18 illustrates the percentages of presence of k-mers in the genome that must be achieved to detect the genome sequence, these percentages being dependent on the length of said sequence;

FIGS. 19A and 19B illustrate the AUC of a lasso prediction and a cluster-lasso prediction as a function of the sequencing depth of coverage for Klebsiella pneumoniae strain isolates; and

FIGS. 20 and 21 illustrate the AUC of a cluster-lasso prediction as a function of the sequencing depth of coverage of metagenomic sample comprising Klebsiella pneumoniae strains.

### DETAILED DESCRIPTION OF THE INVENTION

### A. Embodiment of the Invention

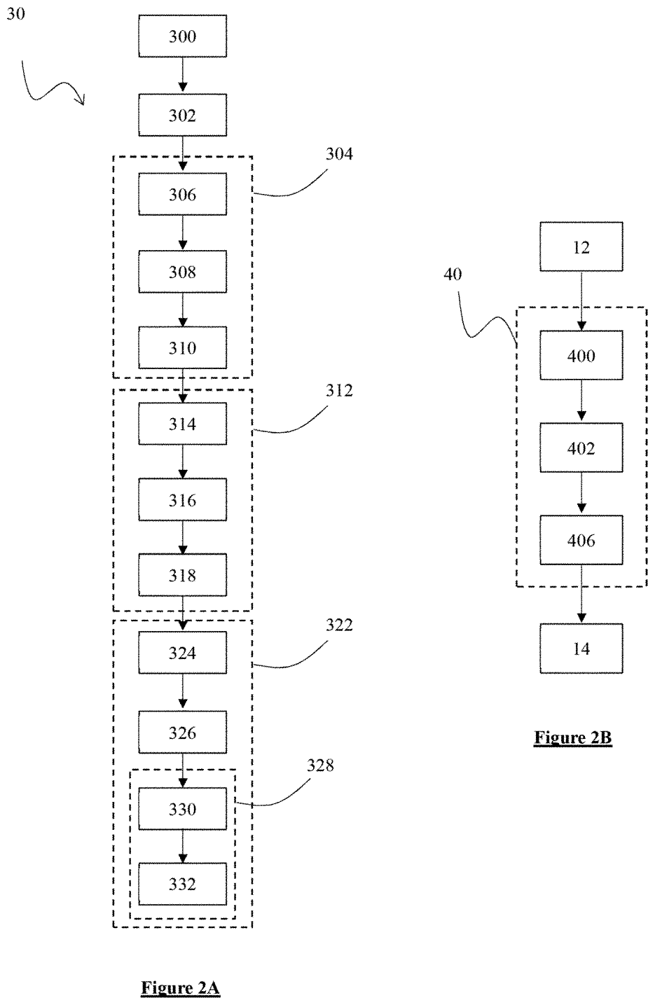

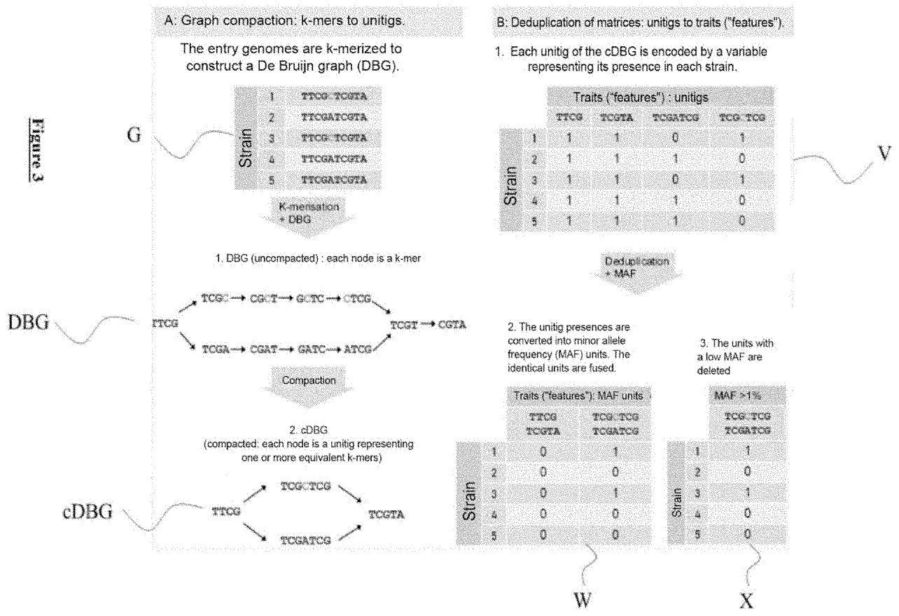

With reference to FIGS. 2A and 2B, a process according to the invention includes a first part 30 of training a model for predicting the susceptibility of a bacterial strain, belonging to a given bacterial species, to an antibiotic as a function of the bacterial genome of said strain, and a second part 40 of applying said model to a strain of said bacterial species to predict its unknown susceptibility.

The first part 30 begins by establishing, in 300, a genomic and phenotypic database for said species. In particular, a set of strains is collected, for example from patients infected with said species, and each collected strain is sequenced to obtain its complete genome, for example using an MiSeq sequencing platform from the company Illumina, and an antibiogram is established to determine its susceptibility to the antibiotic—resistant (“R”), intermediate (“I”) or sensitive (“S”) in accordance with the critical concentrations (or “breakpoints”) of the CLSI standard or the EUCAST standard—for example using a Vitek 2 from the company bioMerieux. Preferably, the genomes take the form of assembled sequences (or “contigs”) produced by digitally assembling digital sequences produced by the sequencing platform (or “reads”) in a manner known per se. The complete digital genome and antibiotic susceptibility of each strain are stored in a computer database to form a learning data set and a test data set.

Advantageously, but optionally, the “resistant” and “intermediate” states are merged so as to obtain two antibiotic susceptibility states. In this way, a binary classification problem is defined which distinguishes between sensitive (“S”) bacterial strains and non-sensitive (“NS”) bacterial strains. For example, the S state is coded with the number 0 and the NS state is coded with the number 1.

The remainder of step 30 is performed by computer and begins, in 302, by clustering the database into two, so as to obtain a learning database and a test database. Preferably, the cluster is a “10-fold” cluster, with nine-tenths of the database constituting the learning database and the remaining tenth constituting the test database. The total number of bacterial strains used for the learning database is noted hereinbelow as N.

In the next step 304, a set of descriptive variables of the bacterial genome is determined. With reference to FIG. 3 in parallel to FIG. 2, the genomes G of the learning database are first described in k-mers, in 306, of size k between 15 and 50, so as to optimally capture the genetic variability of the bacterial species while at the same time limiting the redundancy of the k-mers, for example k=31. In a subsequent step 308, a transformation of the k-mers into a set of distinct genome sequences, without loss of information, is performed. For this purpose, the k-mers are first transformed into a De Bruijn graph DBG of order k and alphabet A, T, G, C. The DBG graph is then transformed into a compacted De Bruijn graph cDBG by automatically clustering the linear paths, and thus having no branching. The k-mers are thus encoded in a graph whose nodes, which are different from each other, correspond to sequences of variable length, these nodes (and equivalently, these sequences) being called “unitigs”.

In a subsequent step 310, the unitigs are clustered into units by deduplication, the minor allele frequency (MAF) of which units is optionally higher than a predetermined threshold “S_{MAF}” of between 98% and 99.5%, for example 99%. In particular:
a. each unitig of the compacted graph cDBG is encoded by a binary variable representing the presence or absence of the unitig in each genome of the database. A matrix V is thus obtained such that its component V_{i,j} is equal to 1 if the j^{th} unitig is present in the i^{th} genome of the database and equal to 0 if it is absent.
b. each vector V₋ⱼ of the matrix V, i.e. each column associated with a unitig, is then corrected for the purpose of calculating an MAF of the unitig. If the allelic frequency of the j^{th} unitig is greater than 0.5, meaning that it is observed in more than 50% of the genomes in the database

$$( i . e . \frac{1}{n} {\sum }_{i = 1}^{n} {V}_{i , j} > 0.5 ) ,$$

then the column V_{.,j} is transformed such that ∀i, V_{i,j}=|1−V_{i,j}|. This transformation has the advantage of rendering identical two columns of the matrix V that are initially complementary, so that the presence co-occurrence of unitigs is identical to their absence co-occurrence.
c. the matrix V thus transformed is then filtered for identical columns in order to group together into units the unitigs of perfect co-occurrence, i.e. those with identical absence/presence of units in the genomes of the database. For example, if the transformed matrix V has its first column V_{.,l} identical to its second column V_{.,2} one of the columns is deleted and the remaining column codes for the union of the unitigs of the first and second column, thus forming a new genomic unit;
d. units whose frequency is lower than (100−S_{MAF})%, i.e. for S_{MAF}=99%, units such that

$$\frac{{\sum }_{i} {V}_{i , j}}{N} < 0.01 ,$$

are then eliminated, implying the deletion of the corresponding column of the matrix V. The result is a matrix X such that its component X_{i,j} is equal to 1 if the j^{th} MAF unit is present in the i^{th} genome of the database, and equal to 0 if it is absent.

The remaining units, referred to hereinbelow as the “MAF units”, are unique, different from each other, and are subsequently used as variables in the machine learning tools described below. This clustering into units and filtering significantly reduce the redundancy induced by the description of the genomes in k-mers without modifying the intrinsic information value of the MAF units regarding their possible involvement in antibiotic susceptibility. Steps 304-310 are described in greater detail in the thesis by M. Jaillard Dancette, “Vers une cartographie des polymorphismes li\vec{e}s à la résistance aux antimicrobiens [Towards a mapping of polymorphisms related to antimicrobial resistance]”, 2018, and in the article by Jaillard M. et al. “A fast and agnostic method for genome-wide association studies: Bridging the gap between k-mers and genetic events”, PLOS Genetics, 2018, and implemented, for example, using the DBGWAS software developed by M. Jaillard (https://gitlab.com/leoisl/dbgwas).

The learning part 30 continues with a step 312 of clustering the MAF units into a limited number of variables that are both predictive of antibiotic susceptibility and have enhanced biological significance, referred to hereinbelow as “clusters”.

Step 312 begins, in 314, with the selection of MAF units that are predictive of antibiotic susceptibility. Advantageously, this selection is performed using a penalized logistic regression tool of the LASSO type. More particularly, for each value A of a set of positive values {λ₁, λ₂, . . . , λₘ}, the following optimization problem is solved:

$$\hat{β} ( λ ) = {argmin}_{β ∈ {ℝ}^{p + 1}} {\sum }_{i = 1}^{N} ℒ ( {y}_{i} , f ( {X}_{i , .} ) ) + λ {\sum }_{j = 1}^{p} \vert {β}_{j} \vert \qquad ( 1 )$$

$$f ( {X}_{i , .} ) = {β}_{0} + {\sum }_{j = 1}^{p} {β}_{j} {X}_{i , j} \qquad ( 2 )$$

in which relationships:
p is the number of MAF units, and thus the number of columns in the matrix X;
N is the number of bacterial strains used to make the learning database, and thus the number of rows in the matrix X;
yᵢ is the susceptibility to the measured antibiotic of the i^{th} bacterial strain, associated with the i^{th} row of the matrix X, i.e. yᵢ=0 if said strain is sensitive and yᵢ=1 otherwise;
[symbol] is a logistic loss function, quantifying the difference between the measured phenotype yᵢ and the predicted phenotype f(X_{i,.}), for example a squared difference of these two terms or a logistic loss function, such that [symbol] (yᵢ,f(X_{i,.}))=log(1−e^{y}^{ᵢ}^{-f(X}^{_{i,.}}⁾).

Any MAF unit that is activated for any of the values of λ and thus predictive, is chosen, i.e. the j^{th} MAF unit is chosen if ∃k∈{1, 2, . . . , m}\ [symbol] (λₖ)≠0. As is known, LASSO tools incrementally activate their variables along the regularization path {λ₁, λ₂, . . . , λₘ}, with one or more variables being added to those already activated as the path {λ₁, λ₂, . . . , λₘ}, is traversed, to light up a maximum of N variables. The set {λ₁, λ₂, . . . , λₘ} is advantageously chosen so as to obtain activation of a maximum of MAF units. For example, it is calculated according to the method described in the article by Friedman et al., “Regularization Paths for Generalized Linear Models via Coordinate Descent”, Journal of Statistical Software, 2010, or as implemented, for example, by the package glmnet 3.0.2, implemented in R, and available, for example, from the website https://cloud.r-project.org/web/packages/glmnet/index.html. In particular, this method allows the number m of regularization variables λ to be chosen in advance. This number is, for example, chosen equal to 100.

The chosen MAF units are referred to hereinbelow as “active MAF units”, the number of active MAF units being noted pₐ. Their ranks, noted aᵢ (i.e. their column indices in the matrix X), are stored in a set a={a₁, a₂, . . . , a₁, . . . , aₚ_{ₐ}}.

Step 310 then consists, in 316, in identifying the MAF units not chosen in step 314 but which have a minimal co-occurrence rate in the genomes with the active MAF units, and then adding the MAF units thus identified to the list of active MAF units. The co-occurrence rate of two units is advantageously measured by the correlation between their corresponding columns of the matrix X. For this purpose, a matrix G, of dimensions pₐ×p, is calculated such that:

∀(i,j)∈[1,pₐ]×[1,p],G_{i,j}=cor(X_{.,a}_{ᵢ},X_{.,j}) (3)

where cor is, for example, the Bravais-Pearson linear correlation, with X_{.,a}_{ᵢ} and X_{.,j} denoting the aᵢ^{th} and the j^{th} columns of the matrix X, respectively.

Then, any MAF unit having a correlation higher than a predefined threshold s₁ with an active MAF unit is chosen, and added to the list of active MAF units, the set of MAF units thus chosen being referred to hereinbelow as “extended active MAF units”. In other words, the j^{th} MAF unit, j∈[1, p], is chosen if ∃i/G_{i,j}>s₁. s₁ is a number that sets the required co-occurrence rate between the extended active MAF units. It is between 0.5 and 1, preferably between 0.8 and 1, and advantageously between 0.9 and 0.95, for example 0.95. The total number of extended active MAF units, noted pₑ, is then much lower than the initial number p of k-mers (pₐ<pₑ<<p), of the order of 10³ as opposed to 105 to 10⁶. The ranks of the extended active MAF units, noted eₗ (i.e. their column indices in the matrix X), are stored in a set e={e₁, e₂, . . . , eₗ, . . . eₚ_{ₑ}}.

In a subsequent step 318, groups, or “clusters”, of highly co-occurring extended active MAF units are explicitly defined by implementing a clustering analysis tool. Preferably, hierarchical clustering is implemented based on a distance matrix calculated from the co-occurrence rates between the extended active MAF units. The hierarchical clustering is, for example, that described in the article by Bühlmann P. et al., “Correlated variables in regression: Clustering and sparse estimation”, Journal of Statistical Planning and Inference, 2013, or implemented by the “hclust” function of the Stats 3.6.2 package implemented in R and available, for example, from the website https://www.rdocumentation.org/packages/stats/versions/3.6.2/source, using an aggregation criterion based on the closest distance (or “single-linkage”).

More particularly, the distance matrix used by the hierarchical clustering is a matrix D, of dimensions pₑ×pₑ, calculated as follows, where X_{.,e}_{ᵢ} and X_{.,e}_{ⱼ} denote the eᵢ^{th} and the eⱼ^{th} columns of the matrix X, respectively:

∀(i,j)∈[1,pₑ]×[1,pₑ],D_{i,j}=|1−cor(X_{.,e}_{ᵢ},X_{.,e}_{ⱼ})| (4)

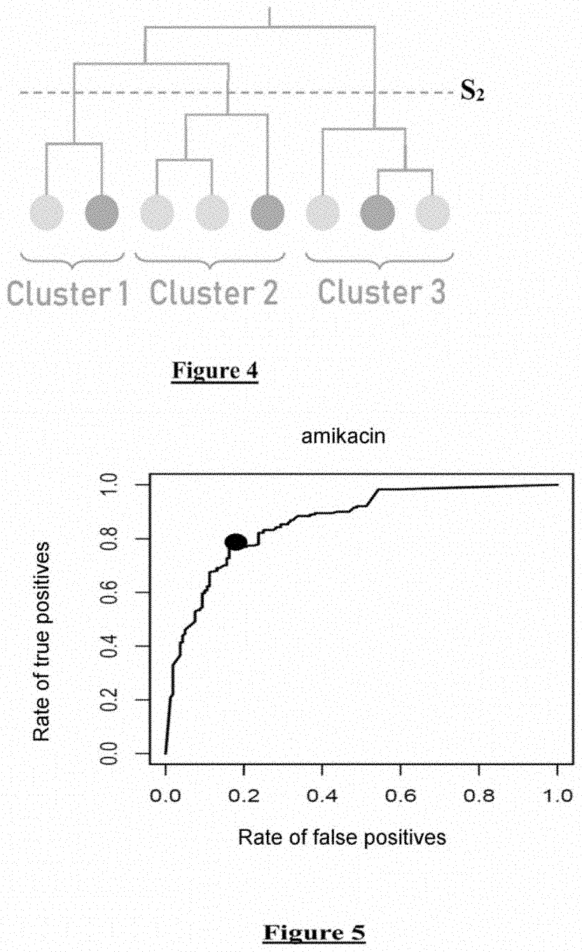

A dendrogram of the extended active MAF units is thus obtained. This dendrogram is then clustered, at a height 1−s₂ as illustrated in FIG. 4, where s₂ is a number between 0 and 1 fixing the co-occurrence rate in the clusters, preferably between 0.5 and 1, preferably between 0.8 and 1, and advantageously between 0.9 and 0.95, for example 0.95. The “lower” part of the dendrogram thus defines clusters in which the extended active MAE units are distributed according to their co-occurrence. Each cluster is also unique and does not share any MAE unit, and in particular any unitigs, with any other cluster. The clusters, being p_{c} in number, are noted C₁, C₂, . . . , Cⱼ, . . . Cₚ_{_{c}} and each cluster Cⱼ is the clustering of pⱼ extended active MAE units of ranks (i.e. their column indices in the matrix X) included in the set c₁ {c_{j,1}, c_{j,2}, . . . , c_{j,l}, . . . , c_{j,p}_{ᵢ}}.

In a subsequent step 320, for each cluster Cⱼ, the unitigs constituting it are broken down into k-mers, with k between 15 and 50, for example k=31, by sliding, in steps of 1, a window of length k over the unitigs. For each cluster Cⱼ, if qⱼ is used to denote the number of unitigs constituting it, qⱼ sets of k-mers respectively associated with the unitigs are stored. For the sake of clarity, the k-mers and unitigs associated with the clusters are called “cluster k-mers” and “cluster unitigs”, respectively.

In a subsequent step 322, a training model for predicting antibiotic susceptibility is used, the variables of which are the clusters C₁, C₂, . . . , Cⱼ, . . . Cₚ_{_{c}}.

Step 322 begins, in 324, by calculating the value of each cluster for each genome in the learning database. Advantageously, this value is equal to the average of the values of the MAF units constituting it. A matrix Y of dimensions N x pₑ, is thus obtained, such that:

$$∀ j ∈ [ 1 , {p}_{c} ] , {Y}_{. , j} = \frac{1}{{p}_{j}} \times {\sum }_{l = 1}^{{p}_{j}} {X}_{. , {c}_{j , l}} \qquad ( 5 )$$

In 326, several prediction models of antibiotic susceptibility are trained from the learning database, with the next step 328 consisting in selecting the one with the best performance according to predetermined criteria.

Advantageously, the prediction models are trained using penalized logistic regression which allows the number of predictive clusters finally retained by the model to be reduced while maintaining a high level of performance. In particular, the prediction models are models according to the following relationships:

$$\{ \begin{matrix}= S if G ( g ) < {S}_{p} \\ = NS if G ( g ) \geq {S}_{p}\end{matrix} \qquad ( 6 )$$

$$G ( g ) = {\hat{B}}_{0} + {\sum }_{j = 1}^{{p}_{c}} {\hat{B}}_{j} {Y}_{{C}_{j}} \qquad ( 7 )$$

in which relationships:
[symbol] is the predicted susceptibility of a bacterial strain belonging to the bacterial species;
g is the genome of said strain;
\hat{B} is a parameter vector of [symbol] ^{p}^{_{c}}⁺¹
Y_{C}_{ⱼ} is the value of the j^{th} cluster Cⱼ for the genome g
Sₚ is a predetermined positive threshold.

Step 326 starts by training the model G using a parsimonious logistic regression tool on the learning database, e.g. a LASSO type penalized regression. In particular, a model G is calculated for each value A of a set of positive values {λ₁^{c}, λ₂^{c}, . . . , λᵢ^{c}, . . . , λ_{M}^{c}} by solving an optimization problem according to the relationship:

$$\hat{B} ( λ ) = {argmin}_{B ∈ {ℝ}^{{p}_{c} + 1}} {\sum }_{i = 1}^{N} ℒ ( {y}_{i} , G ( {Y}_{i , .} ) ) + λ {\sum }_{j = 1}^{{p}_{c}} \vert {B}_{j} \vert \qquad ( 7 )$$

in which relationship:
yᵢ is the susceptibility to the measured antibiotic of the i^{th} bacterial strain, associated with the i^{th} row of the matrix Y, i.e. yᵢ=0 if said strain is sensitive and yᵢ=1 otherwise;
[symbol] is a logistic loss function, quantifying the difference between the measured phenotype yᵢ and the predicted phenotype G(Y_{i,.}), for example a squared difference of these two terms or a logistic loss function, such that [symbol] (yᵢ,f(X_{i,.}))=log(1−e^{y}^{ᵢ}^{-f(x}^{_{i,.}}⁾).

For example, it is calculated according to the method described above in connection with step 314, with a number M=100. 100 models are thus obtained, noted GA and thus 100 prediction models according to the relationships (5)-(6), each depending on the threshold value Sₚ, denoted hereinbelow as G_{λ}_{ᵢ}_{^{c}}(Sₚ).

An estimate of the performance of each prediction model G_{λ}_{ᵢ}_{^{c}}(Sₚ), is then calculated in 328 from the test database. The performance evaluation makes it possible in parallel to calculate the threshold value Sₚ.

In particular, in a step 330, for each genome in the test base, the following are performed:
a. detection of cluster k-mers present in the genome by perfect homology, i.e. a k-mer is detected if it is present in identical form in the genome;
b. detection that a cluster unitig is present in the genome if the percentage of cluster k-mers constituting it, determined as present in the genome in the preceding step, is higher than a first predetermined detection threshold sᵤₙᵢ;
c. calculating an indicator for detecting that an extended MAF unit consisting of several cluster unitigs is present in the genome, defined according to any of the following options:
   i. the indicator is equal to 1 if the percentage of cluster unitigs constituting it, determined to be present in the preceding step, is greater than a second predetermined detection threshold s_{clus}, for example a threshold greater than or equal to 20%, for example 25%. Otherwise, the indicator is equal to 0. This option is the one applied for the examples described below; or
   ii. the indicator is equal to 1 if all the constituent cluster unitigs are determined to be present in the preceding step, and equal to 0 otherwise; or
   iii. the indicator is equal to 1 if at least one constituent cluster unitig is determined to be present in the preceding step, and equal to 0 otherwise.
   iv. the indicator is equal to the percentage of cluster unitigs constituting it which have been detected as present in the preceding step.

Advantageously, the detection threshold sᵤₙᵢ depends on the length of the cluster unitig. In particular, for k between 15 and 50, for example k=31, noting L the length of a cluster unitig for which it is sought to know the presence in the genome, if L≤61 then sᵤₙᵢ=90%, if 61<L≤100 then sᵤₙᵢ=80% and if 100<L then sᵤₙᵢ=70%.

Step 328, in 330, by calculating the value of a cluster as being equal to the mean of the detection indicators of the extended MAF units constituting it, determined in the preceding step. It should be noted that for options i, ii and iii, this mean corresponds to the percentage of extended MAF units detected as present. Once the cluster values have been calculated for all the genomes in the test base, step 330 continues with a model selection strategy that maximizes the sensitivity, specificity and parsimony of the model (i.e. minimizes the number of clusters actually used to predict susceptibility). To do this, for each model G_{λ}_{ᵢ}_{^{c}}(Sₚ), the threshold Sₚ is varied, and for each value of the threshold Sₚ the sensitivity and specificity are calculated according to the relationships:

$$sensitivity = \frac{T P}{T P + F N} \qquad ( 7 )$$

$$specificity = \frac{T N}{F P + T N} \qquad ( 8 )$$

where TP, TN, FP, and FN are the numbers of true positives, true negatives, false positives, and false negatives described in Table 1, respectively.

TABLE 1

Real condition yᵢ

| | | NS | S |
|---|---|---|---|
| Prediction [symbol] | NS | TP | FP |
| | S | FN | TN |

The values of sensitivity and of (1 minus the specificity) for the threshold values Sₚ are then plotted on an ROC curve on the y-axis and x-axis respectively, and the area under the ROC curve, denoted “AUC”, is calculated and stored.

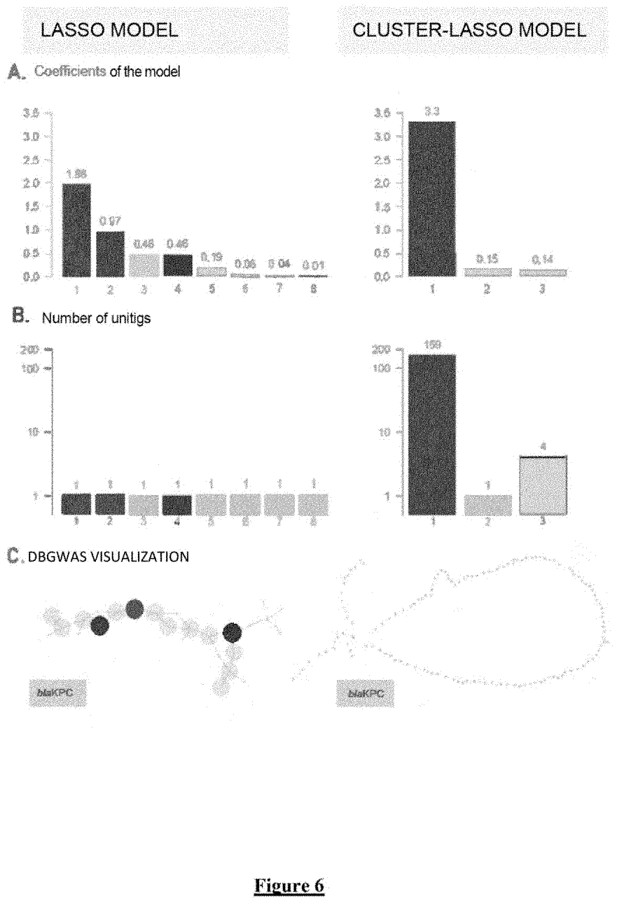

An optimal value [symbol] of the threshold Sₚ for the model G_{λ}_{ᵢ}_{^{c}}(Sₚ) is then calculated as the one corresponding to the point on the ROC curve closest to the point of abscissa 0 and ordinate 1, as illustrated in FIG. 5. The balanced accuracy (“bACC”) is then calculated for the model G_{λ}_{ᵢ}_{^{c}}( [symbol] ) according to the following relationship, the bACC, sensitivity and specificity for the model G_{λ}_{ᵢ}_{^{c}}( [symbol] ) are stored:

$$b A C C = \frac{sensibility + specificity}{2} \qquad ( 9 )$$

The model finally retained among the models G_{λ}_{ᵢ}_{^{c}}( [symbol] ) is the most parsimonious model maximizing the bACC to within one tolerance, for example the model such that:
a. A is the set of models G_{λ}_{ᵢ}_{^{c}}( [symbol] ) such that their bACC is greater than max(bACC)−0.01, or max(bACC) is the maximum of the calculated bACCs;
b. the chosen model is the most parsimonious model in the set A.

The chosen model is stored in a computer memory for subsequent use 40 consisting in predicting the antibiotic susceptibility for a bacterial strain of the bacterial species. The retained clusters, and thus the constituent unitigs, thus form a genomic signature of antibiotic susceptibility.

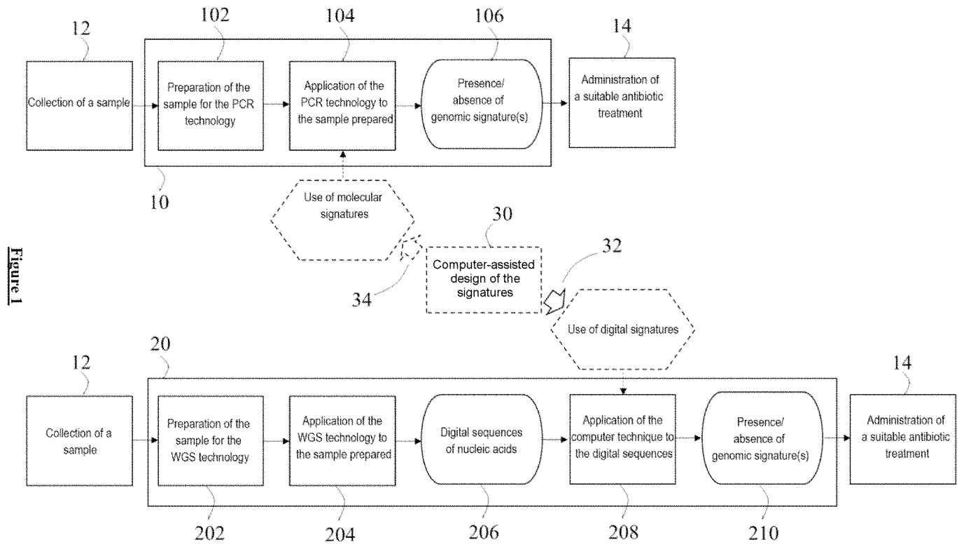

In particular, this prediction consists in:
a. sequencing, in 400, the genome of the bacterial strain by applying a complete genome sequence, for example in the manner described in relation to FIG. 1;
b. calculating, in 402, the value of each cluster in the stored prediction model in the manner described previously;
c. calculating, in 404, the susceptibility [symbol] of the strain using the relationships (5)-(6) for the stored model.

### B. Examples

### B.1. Klebsiella pneumoniae

The process described in FIGS. 2A and 2B was performed for the prediction of the susceptibility to different antibiotics of the bacterial species K. pneumoniae. Table 2 lists the number of strains used for the training and validation of the prediction model, their NS/S phenotypes and the various antibiotics tested.

Table 3 lists the performance of the model trained in accordance with the process according to the invention, known as “cluster-lasso model” or “cluster-lasso”, and the performance of a prediction model trained solely on the basis of a prior art Lasso logistic regression, called “lasso model” or “lasso”. The performance presented in this table corresponds to an estimation by cross-validation, according to the procedure described previously. For the latter, models according to relationships (1) and (2) are calculated, thresholds [symbol] and a final model chosen in the manner described previously for the process according to the invention, and thus on the basis of the same performance criteria.

The “support” columns indicate the number of predictor variables retained for the prediction model, i.e. the number of clusters for which \hat{B}ⱼ≠0 or the cluster-lasso and the number of “active MAF units” for which [symbol] for the lasso. The “unitigs” columns indicate the total number of unitigs retained for the genomic signature, with the number of unitigs in the broadest of the predictor variables in parentheses.

TABLE 2

| | |
|---|---|
| Learning database | Test database |

| | NS | S | NS | S |
|---|---|---|---|---|
| amikacin | 346 | 1319 | 191 | 160 |
| aztreonam | 1426 | 216 | 250 | 10 |
| cefepime | 961 | 608 | 235 | 53 |
| cefoxitin | 976 | 667 | 319 | 138 |
| ceftazidime | 1259 | 136 | 457 | 125 |
| ciprofloxacin | 1461 | 201 | 471 | 137 |
| imipenem | 504 | 1160 | 259 | 301 |
| meropenem | 524 | 1134 | 297 | 86 |
| piper.tazo | 1228 | 432 | 382 | 146 |
| tetracycline | 928 | 737 | 273 | 155 |

TABLE 3

| | Lasso | | | | | Cluster-lasso | | | | |
|---|---|---|---|---|---|---|---|---|---|---|
| | bACC | AUC | support | unitigs | | bACC | AUC | support | unitigs | |
| amikacin | 92.7 | 95.4 | 16 | 22 | (4) | 92.3 | 95.7 | 11 | 93 | (36) |
| aztreonam | 76.7 | 81.9 | 31 | 45 | (3) | 76.9 | 82.3 | 28 | 425 | (125) |
| cefepime | 74.0 | 80.4 | 53 | 65 | (3) | 73.6 | 79.8 | 34 | 385 | (111) |
| cefoxitin | 82.4 | 88.7 | 134 | 155 | (5) | 82.2 | 88.8 | 171 | 1052 | (221) |
| ceftazidime | 91.6 | 95.8 | 51 | 69 | (5) | 90.7 | 95.3 | 43 | 863 | (185) |
| ciprofloxacin | 95.6 | 98.6 | 25 | 27 | (2) | 95.5 | 98.6 | 35 | 422 | (139) |
| imipenem | 93.1 | 93.6 | 10 | 10 | (1) | 92.7 | 93.4 | 7 | 241 | (194) |
| meropenem | 91.7 | 94.0 | 8 | 8 | (1) | 91.4 | 93.5 | 3 | 164 | (159) |
| piper.tazo | 81.6 | 89.6 | 127 | 144 | (4) | 81.5 | 89.0 | 120 | 1220 | (226) |
| tetracycline | 83.0 | 88.5 | 181 | 198 | (3) | 82.9 | 87.7 | 109 | 640 | (104) |

As may be seen, the performance of the cluster-lasso model is similar to that of a prediction model whose learning variables are not constrained by clustering. It is thus noted that the two models show similar performance in terms of balanced accuracy bACC and of AUC, confirming that taking or not taking into account the correlation between the traits has a limited impact in terms of predictive performance. It is also noted that the model support is often slightly smaller with the cluster-lasso (for 8 out of 10 antibiotics), suggesting that several traits chosen separately with the lasso ended up merging into a single cluster through the cluster-lasso. As expected, the total number of unitigs involved in a cluster-lasso model is significantly larger. It is noted that this number is not evenly distributed among the predictive clusters. For example, in the model predicting susceptibility to meropenem, 159 of the 164 unitigs are present in a single cluster, suggesting the presence of a gene as a predictive genomic trait.

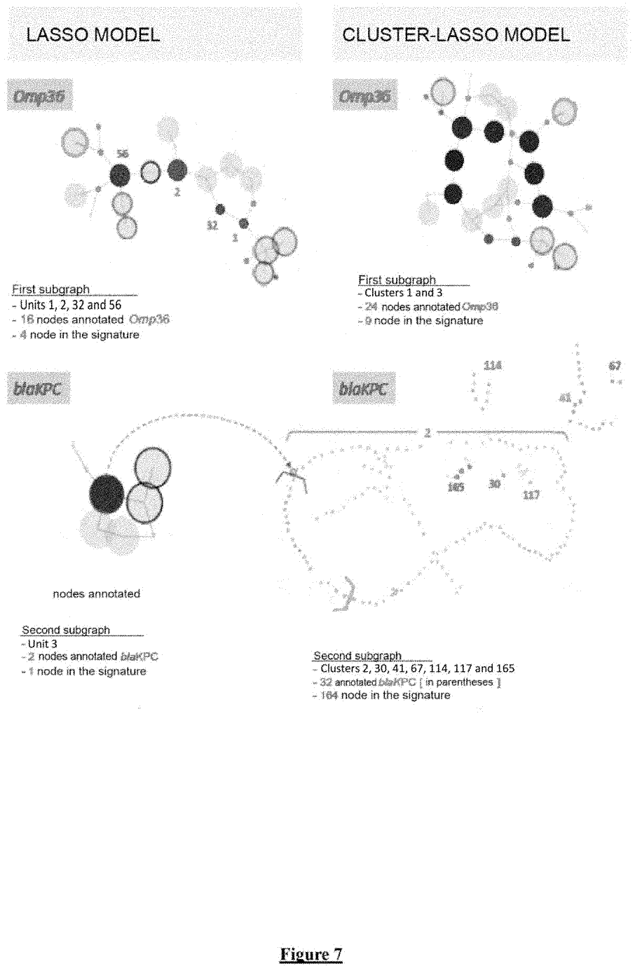

FIG. 6(A) shows the magnitude of the coefficients of the models for meropenem. As is seen, the signature of the cluster-lasso model is essentially summarized by a single important trait, while 4 to 5 traits of the lasso signature have an appreciable weight. It turns out that the cluster with the greatest predictive weight is also the largest cluster in number of unitigs. The visualization of this cluster in the compacted De Bruijn graph cDBG (e.g. using the DBGWAS software, described earlier), as shown in FIG. 6(C), shows that the unitigs of this cluster form a long linear path in the graph. This thus suggests that this cluster corresponds to an entire gene. The annotation of this linear pathway, provided by the DBGWAS software, indicates that it corresponds to the blaKPC gene, which is also well documented in the literature for its role in meropenem resistance. The visualization obtained for the lasso signature indicates, conversely, that three of the eight predictor variables—variables 1, 2 and 4—are also colocated in a region annotated as being the blaKPC gene. However, the fact that the lasso chose these specific unitigs within the blaKPC gene suggests that the resistance determinants involved are point mutations in this gene, namely SNPs or indels. While the annotation of the gene is the same as that obtained with the cluster-lasso, the interpretation of the signature in terms of genetic variants is therefore radically different. More in-depth examination of the lasso signature reveals that the three variables located in the blaKPC gene are in fact highly correlated. By explicitly detecting, according to the invention, that these entities are correlated and merging them into a cluster, along with other correlated genomic units that are not even involved in the lasso signature, the cluster-lasso thus leads to a more biologically meaningful interpretation of the underlying prediction model in two respects. Firstly, as regards the nature of the genomic determinant involved: acquisition or mutation within a gene. Secondly, in terms of its overall contribution to predicting susceptibility, by summing the contributions of several distinct but correlated traits involved in the lasso signature.

Similarly, FIG. 7 illustrates the interpretability of the two prediction models for cefoxitin. Focusing on the subgraphs of the cDGB graph in which the two most predictive clusters are located, the annotation of these two regions identifies the same resistance genes for both methods (firstly, the OmpK36 gene, known to be involved in efflux pumps, and secondly the blaKPC gene). On the other hand, the nature of the genomic determinant (gene presence, SNP, indel, etc.) cannot be deduced from the lasso signature.

The interpretability can even be very detailed. For example, as regards the OmpK36 annotated subgraph obtained for the cluster-lasso signature (top right panel of FIG. 7), it comprises two clusters (clusters 1 and 3), clustering 9 unitigs. These unitigs show a topology attributable to a local polymorphism, namely a complex bubble, with a fork separating the sensitive and resistant strains as described in the article by Jaillard M. et al. “A fast and agnostic method for genome-wide association studies: Bridging the gap between k-mers and genetic events”, PLOS Genetics, 2018. In contrast, the corresponding subgraph obtained for the lasso (top left panel of FIG. 7) includes four units (units 1, 2, 32 and 56) with four distinct values of [symbol] . The distinct values of [symbol] may lead to erroneous conclusions regarding the individual importance of the corresponding unitig sequences. Indeed, when considering a multiple alignment incorporating additional annotated sequences of OmpK36, it appears that active MAF units 2 and 56 represent the wild type and that units 1 and 32 align on the same two amino acid insertion in the L3 loop, as described in the article by Novais A. et al., “Spread of an OmpK36-modified STJS Klebsiella pneumoniae variant during an outbreak involving multiple carbapenem-resistant Enterobacteriaceae species and clones”, European Journal of Clinical Microbiology and Infectious Diseases, 2012. The invention instead provides mean Bⱼ values for each haplotype.

The second subgraph obtained for the lasso signature (bottom left panel of FIG. 7) comprises only one signature trait (shown in black) and seven surrounding nodes (shown in gray), two of which are annotated blaKPC. As the single signature node is not itself annotated, the subgraph could be interpreted as a local polymorphism in the promoter region of the blaKPC gene. However, the cluster-lasso subgraph (bottom right panel of FIG. 7) indicates that this single unitig was chosen by the lasso from hundreds of highly correlated unitigs: they all belong to cluster 2, which includes the complete blaKPC gene (shown in parentheses) and the plasmid sequences in which it is inserted that are highly co-occurring with the gene sequences.

The additional information provided by the cluster-lasso thus makes it possible to conclude that the first causal variable of cefoxitin resistance is a local mutation in the OmpK36 gene. Advantageously, molecular technologies (PCR, NGS, etc.) for predicting cefoxitin resistance will specifically target this mutation. Furthermore, the second causal variable is the acquisition of a complete blaKPC gene, and any DNA sequence specific to blaKPC may advantageously be used by such technologies to predict cefoxitin resistance.

Other bacterial species/antibiotic pairs were tested. Without going into great detail in relation to Klebsiella pneumoniae, the following is described for Salmonella species, Staphylococcus aureus and Neisseria gonorrhoeae:
a first table and a second table similar to Tables 2 and 3 above respectively;
a first, second and third figure illustrating respectively, for the antibiotic under consideration, the absolute values of the coefficients of the lasso model, the absolute values of the coefficients of the cluster-lasso model, and the number of unitigs included in the first 10 most predictive clusters of the cluster-lasso model;
a figure illustrating:
   in its left-hand side, the subgraph(s) of the compacted cDBG graph, involving the most predictive extended MAF units of the lasso model. The subgraphs are first identified by the most predictive units and when other units are present in the subgraph, they are also represented;
   in its right-hand side, the subgraph(s) of the compacted cDBG graph involving the most predictive clusters of the cluster-lasso model. The subgraphs are first identified by the most predictive clusters and when other clusters are present in the subgraph, they are also represented.

### B.2. Salmonella

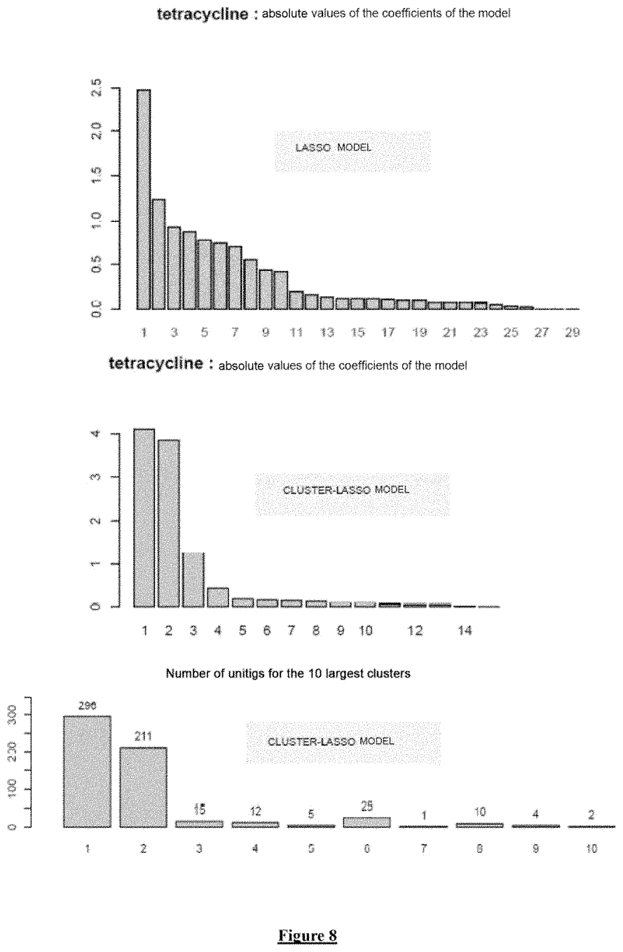

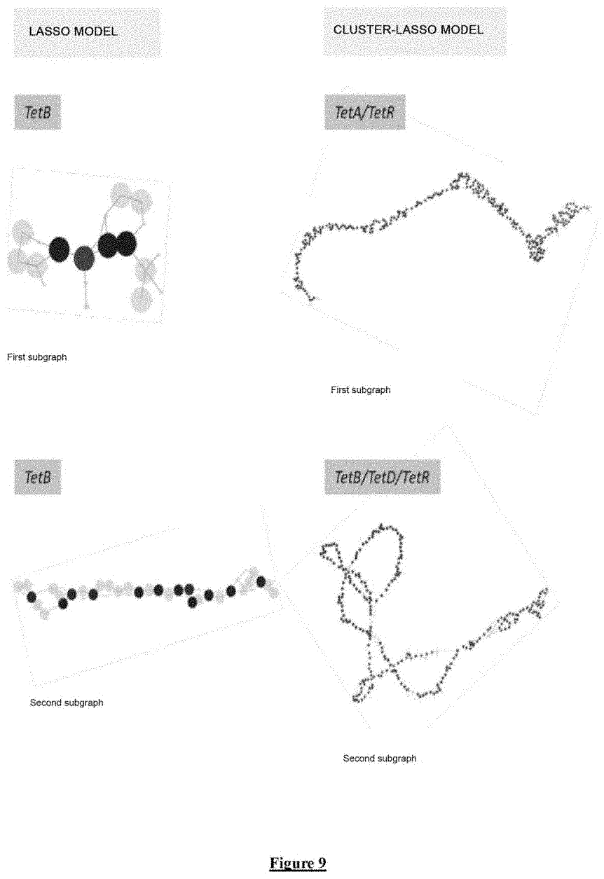

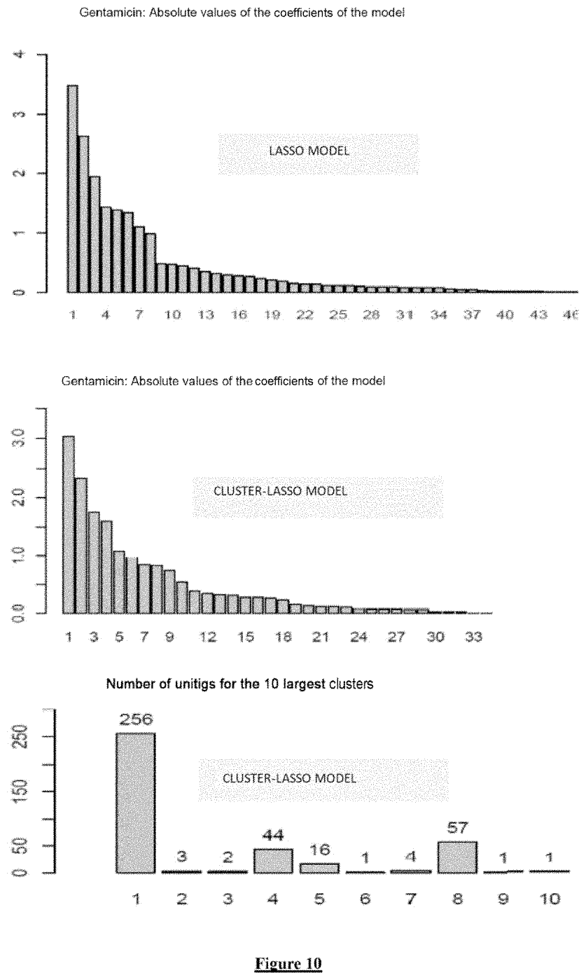

Tables 4 and 5, FIGS. 8 and 9 for tetracycline, FIGS. 10 and 11 for gentamicin.

Unlike the lasso model, which identifies a possible set of point mutations in the TetB genes for the acquisition of tetracycline resistance, without this being otherwise conclusive in view of the large number of mutations that this would imply, the invention based on cluster-lasso identifies the acquisition of resistance for the presence of the TetA gene (cluster 1), the TetB/TetD genes (cluster 2), as well as the acquisition of the TetR gene.

As regards gentamicin resistance, the invention concludes that the AAC3 gene (cluster 1) is acquired and that the OXA, IMP and TEM genes are involved in the resistance mechanisms, whereas the lasso model fails to identify the OXA and IMP genes.

TABLE 4

Learning database

| | NS | S |
|---|---|---|
| tetracycline | 2597 | 1901 |
| gentamicin | 637 | 3862 |

TABLE 5

| | Lasso | | | Cluster-lasso | | |
|---|---|---|---|---|---|---|
| | bACC | AUC | support | bACC | AUC | support |
| tetracycline | 97.4 | 98.2 | 29 | 97.1 | 98.1 | 15 |
| gentamicin | 96.8 | 98.2 | 48 | 96.5 | 98.2 | 34 |

### B.3. Neisseria gonorrhoeae

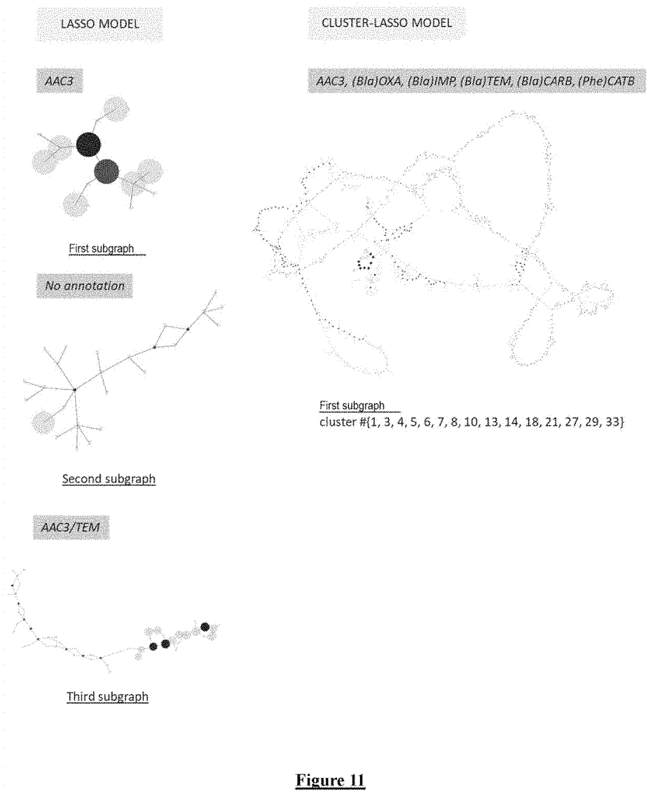

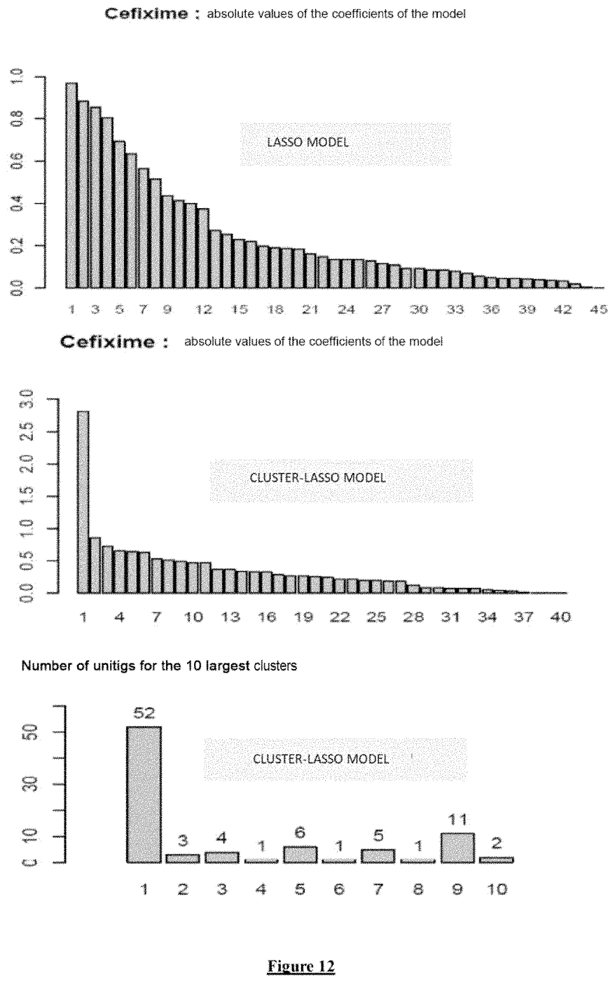

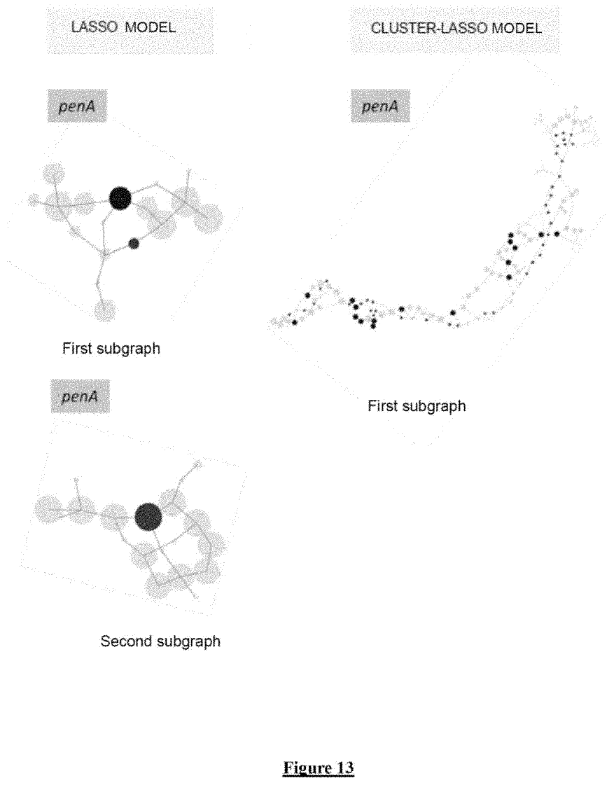

Tables 6 and 7, FIGS. 12 and 13.

As regards cefixime resistance in N. gonorrhoeae, the invention identifies the acquisition of several recombinations in the penM gene.

TABLE 6

Learning database

| | NS | S |
|---|---|---|
| cefixime | 110 | 554 |

TABLE 7

| | Lasso | | | Cluster-lasso | | |
|---|---|---|---|---|---|---|
| | bACC | AUC | support | bACC | AUC | support |
| cefixime | 91.7 | 97.2 | 45 | 92.1 | 97.5 | 40 |

Staphylococcus aureus

Tables 8 and 9, FIGS. 14 and 15.

As regards tetracycline resistance in S. aureus, the invention identifies the acquisition of the TetK gene (cluster 1), but rules out the acquisition of the TetM gene (clusters 2 and 3) as being a highly predictive genomic determinant of gentamicin resistance in view of the low coefficients of the clusters involved, unlike the lasso model, which interprets the TetM gene as highly predictive.

TABLE 8

Learning database

| | NS | S |
|---|---|---|
| tetracycline | 27 | 468 |

TABLE 9

| | Lasso | | | Cluster-lasso | | |
|---|---|---|---|---|---|---|
| | bACC | AUC | support | bACC | AUC | support |
| tetracycline | 96.9 | 96.7 | 12 | 98.9 | 99.6 | 10 |

### C. Computer Means for Performing the Invention

Steps 302, 304, 312, 320, just like steps 60 and 80 described below, are performed by computer, for example a computer unit comprising one or more processors, storage space and random-access memories, capable of storing computer instructions which, when executed, perform the calculations described previously. The computing unit is, for example, a personal computer, a server, or a computing cluster. Similarly, steps 402, 404 are performed by computer, for example a computer unit as described previously. The unit of steps 302, 304, 312, 320 and the unit of steps 402, 404 are different or are the same unit. Advantageously, the predicted susceptibility is displayed on a computer screen, stored in a laboratory or hospital computer system to supplement a patient's record when the bacterial strain infects a patient, or is transmitted to a clinician's mobile device, for example a smartphone.

### D. Extension of the Teaching of the Embodiment of the Invention

### D.1. As Regards the Detection of the Presence of k-Mers, Unitigs and MAF Units in the Bacterial Genome—Step 330

Step 330 describes a way of detecting the presence or absence of a genome sequence, in particular a unitig, or a set of genome sequences in a bacterial genome, in particular a set of units clustering unitigs. In general, the embodiment addresses the issue of whether a sequence or set of sequences should be detected identically in the genome or whether it is possible to accept a certain level of difference between the sequence or group of sequences and the sequences or groups of sequences in the genome in order to decide on their presence or absence. In particular, as explained in the preamble, perfect homology assumes that the learning data is complete to encompass all the variability of the biological species, which is de facto difficult, notably in view of the plasticity of their genome.

Furthermore, the sequenced genome can be tainted by error, notably when it is in the form of “reads”, i.e. sequences produced at the output of sequencing platforms before any bioinformatics processing such as consensus assembly or filtering of poor-quality reads. In this case, a sequence, although present in the genome, may be detected as absent due to sequencing errors and vice versa. In particular, bioinformatics processing generally includes filtering of low-quality reads, and optionally consensus assembly of the filtered reads to obtain assembled sequences, or “contigs”. The optional nature of the assembly generally depends on the context in which the sample analysis is performed. The effect of assembly is to significantly reduce sequencing errors in contigs, at the present time to a level of 10⁻⁵ for SBS technologies used in the platforms of the company Illumina Inc. and to a level of 10⁻² for nanopore technologies used in the platforms of the company Oxford Nanopore Technologies Ltd. On the other hand, since assembly requires high computing power and time, it is not very well suited to “POC” (“point of care”) genomic applications where the computing environment is generally not very powerful and/or to fast, or even real-time applications. In this context, genomic analysis, for example to determine the identity of the one or more species present in the sample and/or their susceptibility to one or more antibiotics as described previously, is performed directly on the filtered or unfiltered reads. However, sequencing errors are of the order of 2 to 3% for SBS technologies and up to 12% for nanopore technologies. Without special precautions, genomic analysis can lead to an equally high error rate.

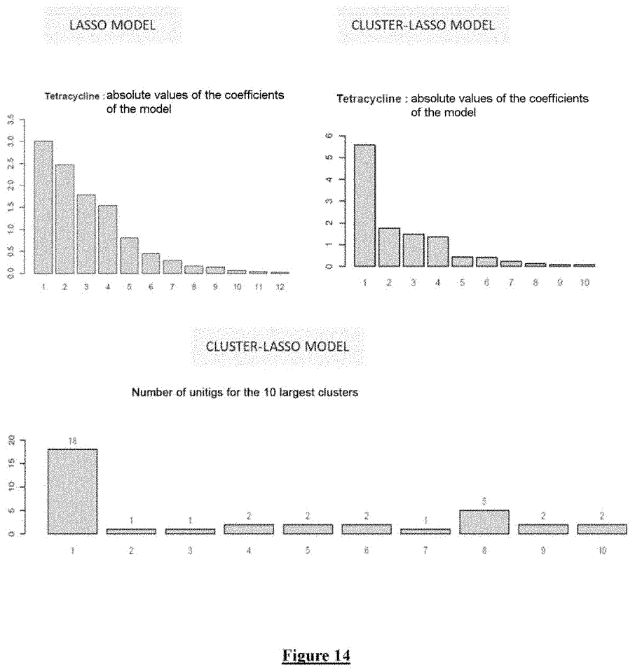

FIG. 16 illustrates a process 50 directed toward more robustly detecting the presence or absence of a genome sequence in a microorganism genome, notably a bacterial strain, a yeast strain or a mold strain, to account for genomic variability and sequencing errors. This process, although independent per se of the processes of FIGS. 2A and 2B, is advantageously performed in step 330 and/or step 402 thereof.

The process 50 comprises a step 70 of sequencing a sample comprising one or more microorganism strains, in the text hereinbelow, and by way of example only, one or more bacterial strains, and pre-processing the reads produced by the sequencing platform, and a step 80 of detecting a predetermined genome sequence in the genome of one of the bacterial strains. This step 80 optionally includes the detection of at least one predetermined set of genome sequences in said genome.

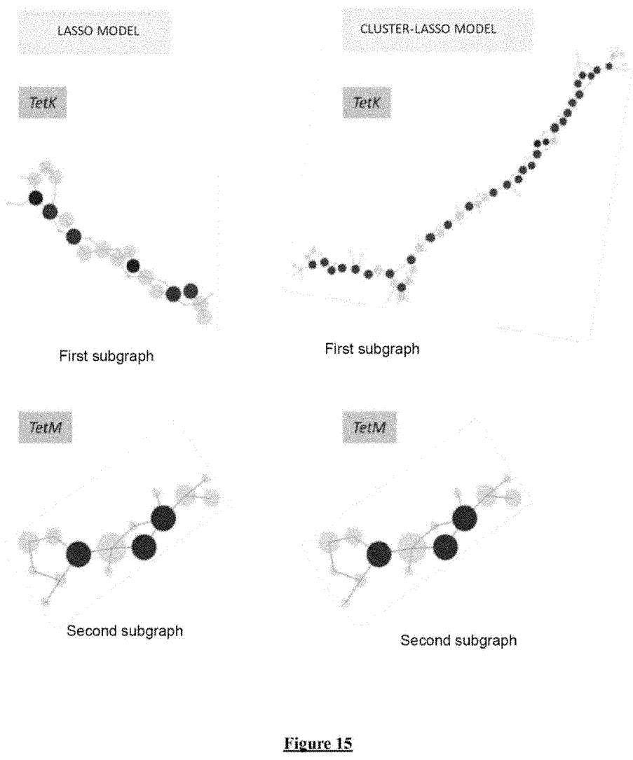

Step 80 uses a breakdown of the genome sequence, and also a certain number of parameters, calculated in a step 60, for example performed prior to the process 50, and stored in a database DB. More particularly, with reference to FIG. 17, step 60 begins with a step 600 in which the genome sequence, noted “SEQ”, is broken down into k-mers of constant length k, with k between 15 and 50, for example k=31, by sliding, in constant steps, preferentially with a step of 1, a window W of length k over the sequence SEQ. At each position of the window W, a k-mer is thus stored. For a sequence SEQ of length L, (L−k+1) k-mers are thus produced. In a subsequent optional step 602, the set of k-mers produced is filtered of its possible duplicates to keep only a set consisting of unique k-mers, noted KM={km₁, . . . , kmᵢ, . . . , kmₛ}, this set forming the breakdown of the sequence SEQ stored in the DB.

Step 70 involves, as is known per se, and for example described in relation to FIG. 1:
in a step 700 preparing the sample for sequencing of the DNA contained in the sample and sequencing the prepared DNA, so that reads are produced and stored;
in a step 702 performing bioinformatics processing generally comprising filtering out of low-quality reads, and optionally assembling the filtered reads by consensus so as to obtain and store assembled sequences, or “contigs”.

The step 80 of detection of the genome sequence SEQ starts with a first test, in 800, which consists in knowing if this detection is performed on contigs or reads. If the detection is performed on contigs, i.e. genome sequences whose error rate is low enough to allow the use of perfect homology through the detection of k-mers, the process continues, in 802, by detecting the presence or absence of each k-mer kmᵢ of the KM set in the contigs. In particular, the k-mer kmᵢ is detected if it is identically present in at least one of the contigs.

In a subsequent step 804, a test is performed to determine whether the sequence SEQ should be detected identically in the bacterial genome. If so, the sequence SEQ is detected, in 806, if all the k-mers kmᵢ in the KM set are detected as being present in the contigs. It is noted in this regard that due to the sequencing technology and/or the assembly technology, the sequence SEQ does not necessarily end up whole in a contig, but may instead be split between several contigs, with the probability of such splitting increasing with the length L of the sequence SEQ. The breakdown into k-mers thus makes it possible to identify the latter in the genome even if it is not present as such in the contigs.

If the sequence SEQ is not sought in an identical manner in 804, the genome is searched in 808 for at least the sequence SEQ or one of its variants. These variants correspond, for example, to mutations in the original sequence SEQ or to imperfect identification of the latter. As described above, the sequence SEQ may be the product of identification of a genetic determinant (e.g. of resistance, virulence, identity, etc.) based on a priori data or knowledge. If the latter is imperfect, the sequence SEQ may not reflect the full diversity of said determinant. By permitting the identification of the sequence SEQ or one of its variants, the process allows the initial imperfection of the data and knowledge to be corrected and thus the genetic determinant to be detected. To a lesser extent, it also allows possible residual sequencing errors in the contigs to be taken into account. Furthermore, independently of the error correction, it also makes it possible, from a single sequence SEQ, to detect whether at least one member of a set of variants is present in the genome, without having to perfectly detect each of said variants.

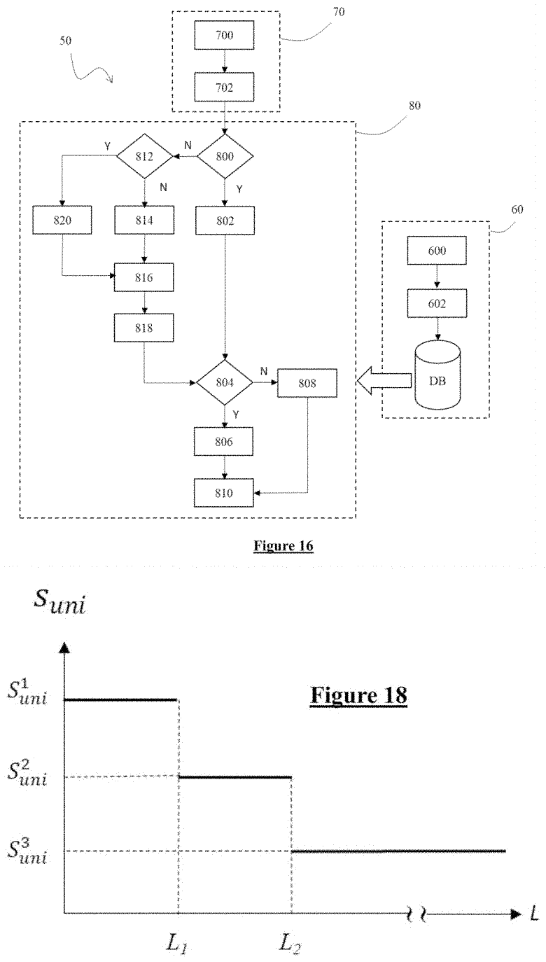

In a first variant, in 808, the sequence SEQ or one of its variants is detected if the percentage of its constituent k-mers is greater than a predetermined threshold sᵤₙᵢ, for example greater than 70%. In a preferred variant, this percentage is dependent on the length L of the sequence SEQ and more particularly decreases as a function of L. Specifically, it is preferable to keep a sufficiently long length of k-mers, in particular greater than 15, and preferably greater than 30, so that the latter remain specific to the sequence SEQ. Thus, as the length L decreases, a difference with the sequence SEQ is found in an increasingly large percentage of k-mers, which makes it possible to correct for the percentage sᵤₙᵢ decreasing as a function of the length L. As illustrated in FIG. 18, the percentage sᵤₙᵢ is, for example, decreasing by piece, and comprises three values. Advantageously, for k between 15 and 50, for example k=31, if L≤61 then sᵤₙᵢ=90%, if 61<L≤100 then sᵤₙᵢ=80% and if 100<L then sᵤₙᵢ=70%.

Once the sequence SEQ has been detected, optionally, the process continues, at 810, with the detection of a set of genome sequences, denoted {SEQ₁, . . . , SEQᵢ, . . . SEQ_{E}}, in a manner described below.

If the detection of the sequence SEQ is performed on reads (test 800), and thus before any bioinformatics processing correcting sequencing errors, the process takes these errors into account to accurately detect the presence/absence of a k-mer in the reads, and thus in the genome. The advantage of detecting directly from the reads lies in the speed of data processing, which is less than 2-3 minutes for a given computational environment, whereas assembly alone can take over an hour for the same environment.

In a first variant, a k-mer is detected if the reads contain a minimum number of copies of said k-mer, for example a number greater than or equal to 3. However, this variant has the drawback of not taking into account the sequencing depth of coverage. To a first approximation, the sequencing errors are distributed over the entire genome and as such the greater the sequencing depth of coverage, the higher the probability of detecting the k-mer. However, due to sequencing errors, it is difficult to ascertain whether the k-mer is actually present in the genome or whether the k-mer detected in the reads is the product of another k-mer bearing sequencing errors. In a preferred variant, detection depends on the actual sequencing depth of coverage for the bacterial strain in which the sequence SEQ is sought.

To this end, a test 812 is performed to determine whether the sample is a metagenomic sample, or more generally a sample containing several different species (several bacterial species, human DNA, or other), or a sample prepared from an isolate of the bacterial strain. In the latter case, since only one strain is present, all reads belong to said strain and the sequencing depth of coverage, noted “cov”, is calculated, in 814, for example according to the relationship:

$$cov = \frac{{N}_{r}}{{N}_{g}} \qquad ( 10 )$$

in which relationship N, is the total number of bases included in the reads and N. is the number of bases in a reference genome of the bacterial species to which the bacterial strain belongs, preferentially having a mean size or close to the mean of the genome sizes observed for said species (e.g. Ng=4.4 million base pairs (Mbp) for Mycobacterium tuberculosis).

A copy number, noted N_{cov}, which needs to be detected in the reads to affirm that a k-mer is indeed present in the genome, is then calculated in 816 according to the following relationship:

N_{cov}=τ×cov (11)

where τ is a predetermined parameter, preferentially taking into account the sequencing error rate of the sequencing technology used, advantageously between 5% and 15%, preferably greater than or equal to 10%, for example 10%. For the latter percentage and a depth of 100, 10 identical copies of a k-mer must therefore be detected to determine that it is actually present in the reads. A rate of 10% notably enables the presence of a k-mer to be accurately detected in reads produced by a GridION platform from the company Oxford Nanopore Technology Ltd using the R9.4 library preparation kit from the same company. This rate also allows precision detection in reads produced by SBS-type sequencing technology, for example the MiSeq platform from the company Illumina Inc.

Detection of the presence or absence of each k-mer kmᵢ of the KM set in the reads is then performed in 818. In particular, the k-mer kmᵢ is detected if there are at least N_{cov} identical copies in the reads. The process then continues with step 804 described previously.

If the sample comprises several species (test 812), the process consists in determining the sequencing depth of coverage for the bacterial species under consideration. More particularly, “taxonomic binning” is performed in 820, such binning consisting in assigning to each read an origin among the species present in the sample. This type of binning is well known in the prior art and uses, for example, the classification described in the article by Wood D. E. et al., “Kraken: ultrafast metagenome sequence classification using exact alignments”, Genome Biology, 2014, or as performed by the “Kraken2” software downloadable from the website https://github.com/DerrickWood/kraken2/releases.

The sequencing depth of coverage of said bacterial species under consideration is then calculated, for example according to either of the following relationships:

$$cov = \frac{{N}_{r}^{m}}{{N}_{g}} \qquad ( 12 )$$

$$cov = ρ \times \frac{{N}_{r}}{{N}_{g}} \qquad ( 13 )$$

in which relationship Nᵣ^{m} is the total number of bases included in the reads assigned to the bacterial species to which the bacterial strain under consideration belongs, and N. is the number of bases in a medium-sized genome of the bacterial species, and p is the relative proportion of the bacterial species in the sample. This relative proportion is calculated, for example, using the classification described in the article by Wood D. E. et al., “Kraken: ultrafast metagenome sequence classification using exact alignments”, Genome Biology, 2014, or as performed by the “Kraken2” software downloadable from the website https://github.com/DerrickWood/kraken2/releases. The process continues with step 816 of calculating the copy number as a function of the sequencing depth of coverage just calculated.

Referring again to step 810 of detecting the set of genome sequences, denoted {SEQ₁, . . . , SEQᵢ, . . . SEQ_{E}}, this step follows the detection of each sequence SEQᵢ in the manner described previously. More particularly, the detection of said set is implemented according to one of the following options:
i. the set is detected as present in the genome if the percentage of SEQᵢ determined as being present is greater than a second predetermined detection threshold s_{clus}, for example a threshold greater than or equal to 20%, e.g. 25%, and absent otherwise; or
ii. the set is detected as present in the genome if all the SEQᵢ are determined as being present, and absent otherwise; or
iii. the set is detected as present in the genome when at least one of the sequences SEQᵢ is determined as being present, and absent otherwise; or
iv. the set is detected as present in the genome with a probability equal to the percentage of SEQᵢ determined as being present.

Firstly, it is noted that the detection performance of sequences SEQ or sets of SEQs is very similar to that obtained directly by k-mer identification, as evidenced by the performance of a cluster-lasso based process compared to the performance of a lasso-based process as described previously.

Secondly, the detection process 50 is robust with respect to the type of sequencing technology employed, and notably to their sequencing errors. The following table illustrates, for 37 test strains of Klebsiella pneumoniae species in isolate form, with variant detection (percentage 70%, 80%, 90%) for unitigs (equivalent to SEQᵢ) and unit detection according to option i) above, the results of a cluster-lasso prediction of resistance to different antibiotics for sequencing by an MiSeq (“Illumina”) and by a GridION (“ONT”). The two sequencing technologies are tested as a function of their reads and the contigs produced by the assembly of their reads. Table 10 shows that the results are similar for both technologies, whereas they have significantly different sequencing error rates, but also similar results for both reads and contigs.

TABLE 10

| | Type of data | bACC | Sensitivity | Specificity | AUC |
|---|---|---|---|---|---|
| Piperacillin | Illumina Reads | 86.5 | 81.5 | 100 | 91.1 |
| | Illumina Contigs | 86.5 | 81.5 | 100 | 92.2 |
| | ONT Reads | 89.2 | 85.2 | 100 | 91.9 |
| | ONT Contigs | 86.5 | 81.5 | 100 | 91.1 |
| Cefepime | Illumina Reads | 94.6 | 92.3 | 100 | 98.3 |
| | Illumina Contigs | 97.3 | 96.2 | 100 | 98.6 |
| | ONT Reads | 94.6 | 92.3 | 100 | 96.5 |
| | ONT Contigs | 97.3 | 96.2 | 100 | 98.3 |
| Meropenem | Illumina Reads | 86.5 | 81.0 | 93.3 | 92.7 |
| | Illumina Contigs | 86.5 | 81.0 | 93.8 | 92.7 |
| | ONT Reads | 78.4 | 61.9 | 100 | 86.9 |
| | ONT Contigs | 86.5 | 81.0 | 93.8 | 92.6 |
| Cipro- | Illumina Reads | 94.6 | 96.0 | 91.7 | 95.7 |
| floxacin | Illumina Contigs | 94.6 | 96.0 | 91.7 | 95.7 |
| | ONT Reads | 91.9 | 88.0 | 100 | 96.7 |
| | ONT Contigs | 94.6 | 96.0 | 91.7 | 95.7 |

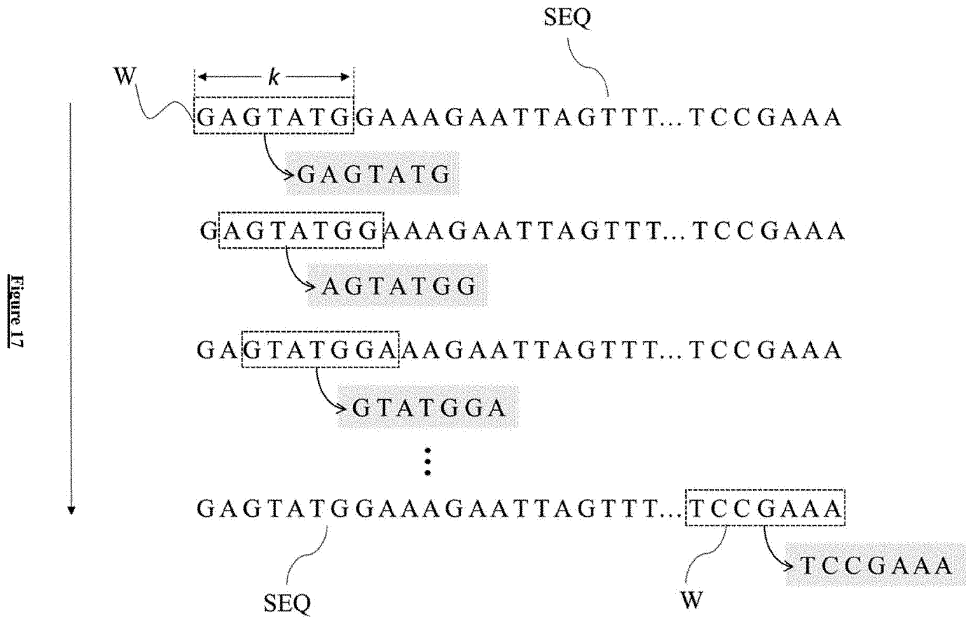

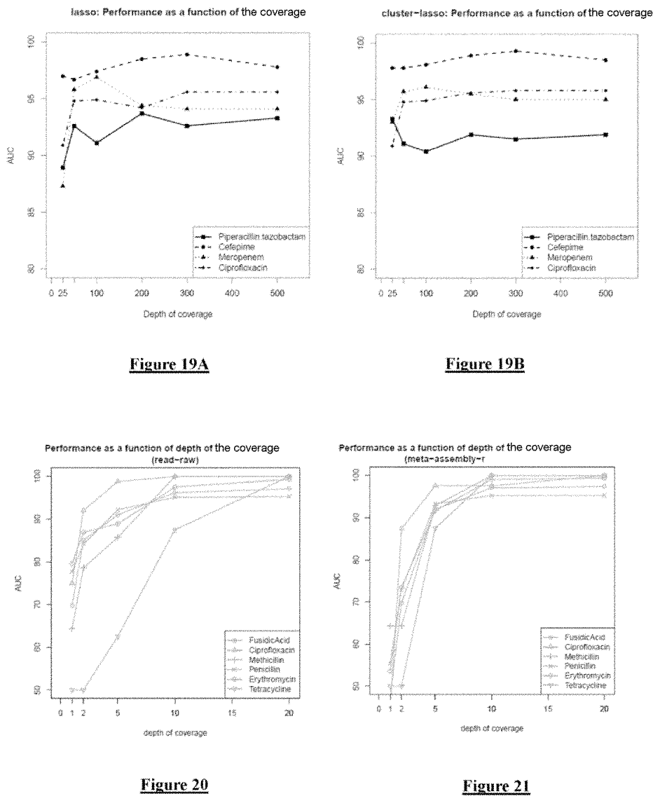

Furthermore, the prediction performance (AUC) as a function of sequencing depth of coverage for the ONT technology is shown in FIGS. 19A (for lasso prediction) and 19B (for cluster lasso prediction), the samples being produced from isolates of Klebsiella pneumoniae strains. It is noted that taking into account the sequencing depth of coverage, and thus an increasing copy number with the latter, makes it possible rapidly to obtain stable performance, from a depth of 30. FIGS. 20 and 21 illustrate, through a simulation study, the effect of a metagenomic sample comprising Staphylococcus aureus strains (here simulating clinical samples from bronchoalveolar lavage) on the performance of a cluster-lasso prediction as a function of reads (FIG. 20) or contigs (FIG. 21) for the Illumina technology. Very fast stabilization at high performance depending on the actual sequencing depth of coverage of the Staphylococcus aureus strains present in the sample is also noted.

### D.2. As Regards Other Features of the Embodiment

A particular embodiment of the invention has been described. This process may be modified in accordance with the following features, taken alone or in combination:
prediction of susceptibility to an antibiotic has been described. The invention applies to any type of phenotype, for example the virulence of a bacterial strain, its ribotype, etc.;
the application of the invention to a biological sample taken from a patient has been described. The invention applies to any type of sample comprising bacteria, in particular a sample taken from an animal or a sample taken from the environment;
bacteria have been described. The invention also applies to yeasts and molds;
the complete sequencing of the bacterial genome has been described. As a variant, the genome sequencing is partial and targets one or more specific regions known to be involved in antibiotic susceptibility;
in the described embodiment, the value of k-mers and unitigs in the genome is binary (absence or presence) as encoded for example in the matrix X. As a variant, the value of k-mers or unitigs is equal to the number of copies thereof in the genome;
binary prediction (S and NS states) has been described. As a variant, the susceptibility is ordinal (higher number of states, e.g. S, R and I) or linear (e.g. prediction of the minimum inhibitory concentration, or “MIC”). In this case, the regression is ordinal or linear;
prediction models trained by logistic regression of the LASSO type have been described. Other parsimonious algorithms are possible, for instance random forest models, gradient boosting, set covering machine, aggregation and Monte-Carlo or deep learning, or any type of penalized Lasso learning (elastic-net, group-lasso, fused lasso, adaptive lasso, etc.);
selection of MAF units using a logistic regression lasso has been described. Other selections are possible, for example one as described in the article by Friedman J. H. “Greedy Function Approximation: A Gradient Boosting Machine”, The Annals of Statistics, 2001, and as used, for example, by the software “xGBoost”, available from the website https://xgboost.readthedocs.io/en/latest/, or any other nonlinear selection;
clustering based on Bravais-Pearson correlation values has been described. Other types of co-occurrence measurements are possible, for example a Jaccard or Sorensen-Dice distance;
a particular clustering has been described. Other types of clustering are possible, for example “standard” hierarchical clustering;
the value of a cluster has been described as being equal to the mean of the units constituting it. Other values are possible. For example, a logistic regression of the “lasso group” type is performed for each of the clusters in order to assign different weights to the different units constituting it;
the use of learning algorithms from assembled genomes has been described. As a variant, the invention applies directly to genomes produced by a sequencing platform, i.e. to genomes in the form of reads, optionally filtered from low quality reads.

1. A process for determining a phenotypic trait of a bacterial strain, involving:
partial or total sequencing of the genome of the strain;
applying to the sequenced genome a predetermined model for predicting the phenotypic trait, the model having groups of genome sequences as variables,
wherein the groups are chosen so that:
a co-occurrence rate in the genome of the bacterial species of the constituent genome sequences of the groups is above a predetermined threshold; and
the groups are clusterings of the genome sequences according to their co-occurrence rates in the genome of the bacterial species.

2. The process as claimed in claim 1, in which a first subset of the genome sequences is a set of unclustered variables of a first phenotypic trait prediction model.

3. The process as claimed in claim 2, in which a second subset of the genome sequences is a set of genome sequences having a co-occurrence rate with the first set greater than a predetermined threshold.

4. The process as claimed in claim 2, in which the first prediction model is a model trained from a database of genomes and phenotypic traits of bacterial strains belonging to the bacterial species, the learning belonging to the class of parsimonious learning.

5. The process as claimed in claim 4, in which the learning is performed by a logistic regression of the LASSO type.

6. The process as claimed in claim 1, in which the genome sequences correspond to nodes of a compacted graph obtained from genome sequences of constant length and occurring in genomes of strains belonging to the bacterial species.

7. The process as claimed in claim 6, in which the compacted graph is calculated by
calculating a De Bruijn graph of alphabet A, T, G, C from the genome sequences of constant length;
compacting linear paths of the De Bruijn graph so as to obtain the compacted graph.

8. The process as claimed in claim 1, in which the clustering of genome sequences involves:
obtaining first groups of genome sequences by applying sequence clustering with perfect co-occurrence in genomes of strains belonging to the bacterial species;
obtaining groups of the prediction model by applying a second clustering of the first groups based on the co-occurrence rates of the first clusters in the genomes.

9. The process as claimed in claim 1, in which the clustering of genome sequences involves:
calculating a dendrogram as a function of co-occurrence rates of genome sequences in genomes of strains belonging to the bacterial species; and
clustering the dendrogram at a predetermined height to obtain the groups of genome sequences.

10. The process as claimed in claim 8, in which the dendrogram is calculated on the first groups of genome sequences.

11. The process as claimed in claim 1, in which the prediction model is trained on a database of genomes and phenotypic traits of bacterial strains belonging to the bacterial species, the learning belonging to the class of parsimonious learning.

12. The process as claimed in claim 11, in which the learning is performed by a LASSO-type logistic regression.

13. The process as claimed in claim 11, in which the value of a group of genome sequences for parsimonious learning is calculated:
for each genome sequence belonging to the group, calculating a vector of occurrences of the sequence in the genomes of the database;
calculating the mean vector of the occurrence vectors, the mean vector constituting the value of the group.

14. The process as claimed in claim 1, in which the value of a group of genome sequences for the application of the prediction model applied to the bacterial strain is equal to the percentage of genome sequences of the group present in the genome of the bacterial strain.

15. The process as claimed in claim 8, in which the value of a group of genome sequences for the application of the prediction model applied to the bacterial strain is equal to the percentage of the first groups of the group present in the genome of the bacterial strain.

16. The process as claimed in claim 8, in which the value of a group of genome sequences for the application of the prediction model applied to the bacterial strain is calculated by:
calculating, for each first group of the group, the percentage of genome sequences of the first group present in the genome of the bacterial strain; and
calculating the value as being equal to the mean of the percentages of the first groups of the group.

17. The process as claimed in claim 16, in which a genome sequence is present in the genome of the bacterial strain when a percentage of genome sequences of constant length constituting the sequence is greater than a predetermined threshold depending on the length of the sequence.

18. A process for identifying genomic signatures predictive of a phenotypic trait of a bacterial species, the process comprising:
building a database of genomes and phenotypic traits of a plurality of bacterial strains belonging to the bacterial species;
calculating a set of genome sequences descriptive of the genomes of the database, and for each of the sequences calculating a vector of occurrence of the sequence in the genomes of the database;
selecting, from the set of genome sequences, sequences having a co-occurrence rate above a predetermined threshold, the rate being calculated as a function of occurrence vectors;
clustering the chosen genome sequences according to their co-occurrence rates, so as to obtain groups of genome sequences;
training a model for predicting the phenotypic trait of the bacterial species to the antibiotic having groups of genome sequences as variables;
identifying the signature as being equal to the union of groups of genome sequences.

19. The process as claimed in claim 18, in which the selection of genome sequences involves:
selecting a first set of genome sequences consisting of unclustered variables of a first prediction model of the phenotypic trait;
selecting, from the genome sequences not chosen in the first selection, genome sequences having a co-occurrence rate with the genome sequences of the first set greater than the predetermined threshold.

20. The process as claimed in claim 18, in which the first prediction model is a model trained from a database of genomes and phenotypic traits of bacterial strains belonging to the bacterial species, the learning belonging to the class of parsimonious learning.

21. The process as claimed in claim 20, in which the learning is performed by a logistic regression of the LASSO type.

22. The process as claimed in claim 18, in which the genome sequences correspond to nodes of a compacted graph obtained from genome sequences of constant length and occurring in genomes of a strain belonging to the bacterial species.

23. The process as claimed in claim 22, in which the compacted graph is calculated by
calculating a De Bruijn graph of alphabet A, T, G, C from the genome sequences of constant length;
compacting linear paths of the De Bruijn graph so as to obtain the compacted graph.

24. The process as claimed in claim 18, in which the clustering of the chosen genome sequences involves:
obtaining first groups of genome sequences by applying sequence clustering with a perfect co-occurrence in genomes of strains belonging to the bacterial species;
obtaining the groups of the prediction model by applying a second clustering according to the co-occurrence rates of the first groups of sequences in the genomes.

25. The process as claimed in claim 18, in which the clustering of chosen genome sequences involves:
calculating a dendrogram as a function of co-occurrence rates of genome sequences in genomes of strains belonging to the bacterial species; and
clustering the dendrogram at a predetermined height to obtain the groups of genome sequences.

26. The process as claimed in claim 24, in which the dendrogram is calculated on the first groups of genome sequences.

27. The process as claimed in claim 18, in which the prediction model is trained on a database of genomes and phenotypic traits of bacterial strains belonging to the bacterial species, the learning belonging to the class of parsimonious learning.

28. The process as claimed in claim 27, in which the learning is performed by a logistic regression.

29. The process as claimed in claim 27, in which the value of a group of genome sequences for logistic regression is calculated by
for each genome sequence belonging to the group, calculating a vector of occurrence of the sequence in the genomes of the database;
calculating the mean vector of the occurrence vectors, the mean vector constituting the value of the group.

30. The process as claimed in claim 18, in which the phenotypic trait is the antibiotic susceptibility of the bacterial strain.

31. A process comprising performing a molecular test for predicting a phenotypic trait using the genomic signature identified according to the process as claimed in claim 18, in which the molecular test at least partially targets the genomic signature.

32. A computer program product storing computer-executable instructions for the application of a model for predicting a phenotypic trait of the a bacterial strain, the application being as claimed in claim 1.

33. A system for determining a phenotypic trait of a bacterial strain, comprising:
a sequencing platform for partial or total sequencing the genome of the strain;
a computer unit configured to apply to the sequenced genome, a predetermined model for predicting the phenotypic trait, as claimed in claim 1.

34. A computer program product storing computer-executable instructions for performing a process for identifying genomic signatures predictive of a phenotypic trait, the process being as claimed in claim 18.
